(19) United States
(12) Patent Application Publication
(10) Pub. No.: US 2023/0407004 A1
(43) Pub. Date: Dec. 21, 2023

(54) METHOD FOR PRODUCING FLUORINE-CONTAINING COMPOUND AND METHOD FOR PRODUCING SURFACE TREATMENT AGENT

(71) Applicant: AGC Inc., Tokyo (JP)

(72) Inventors: Yoshitaka NOMURA, Chiyoda-ku (JP); Makoto UNO, Chiyoda-ku (JP)

(73) Assignee: AGC Inc., Tokyo (JP)

(21) Appl. No.: 18/456,128

(22) Filed: Aug. 25, 2023

Related U.S. Application Data
(63) Continuation of application No. PCT/JP2022/008853, filed on Mar. 2, 2022.

(30) Foreign Application Priority Data
Mar. 5, 2021 (JP) 2021-035323

(51) Int. Cl. C08G 65/337; C08G 65/336; C09K 3/18

(52) U.S. Cl. CPC C08G 65/337; C08G 65/336; C09K 3/18

(57) ABSTRACT

An object is to provide a method for producing a fluorine-containing compound by using an easily available compound under relatively mild reaction conditions to produce a fluorine-containing compound, and a method for producing a surface treatment agent using the fluorine-containing compound obtained by the production method.

A method for producing a fluorine-containing compound represented by the following formula (C1) or (C2), the method including: reacting a compound represented by the following formula (A1) or (A2) with a compound represented by the following formula (B1) or (B2).

G¹-L¹-CR¹R²—X¹ Formula (A1)

X²—CR³R⁴-L²-G²-L³-CR⁵R⁶—X² Formula (A2)

R¹¹—ZnR¹² Formula (B1)

R¹¹—BR¹³R¹⁴ Formula (B2)

G¹-L¹-CR¹R²—R¹¹ Formula (C1)

R¹¹—CR³R⁴-L²-G²-L³-CR⁵R⁶—R¹¹ Formula (C2)
where, each reference sign in the formula is as described in the specification.

### INCORPORATION BY REFERENCE

This application is based upon and claims the benefit of priority from Japanese Patent Application 2021-35323 filed on Mar. 5, 2021, and PCT application No. PCT/JP2022/008853 filed on Mar. 2, 2022, the disclosure of which is incorporated herein in its entirety by reference.

### BACKGROUND

The present invention relates to a method for producing a fluorine-containing compound and a method for producing a surface treatment agent.

Fluorine compounds are used in various fields such as agricultural chemicals, pharmaceuticals, and functional materials, and it is required to synthesize various structures by a simpler method.

Various studies have been made on a method for synthesizing a compound having a structure in which an alkyl group is bonded to a fluoroalkyl group.

For example, Japanese Unexamined Patent Application Publication No. 2018-43940 discloses a method for producing a fluorine-containing compound by adding a perfluoroalkyl bromide to an olefin compound by a radical reaction.

In addition, Teruo Umemoto, “Electrophilic Perfluoroalkylating Agents”, Chem. Rev. 1996, 96, 1757-1777 discloses a compound represented by the following formula as an electrophilic perfluoroalkylating agent.

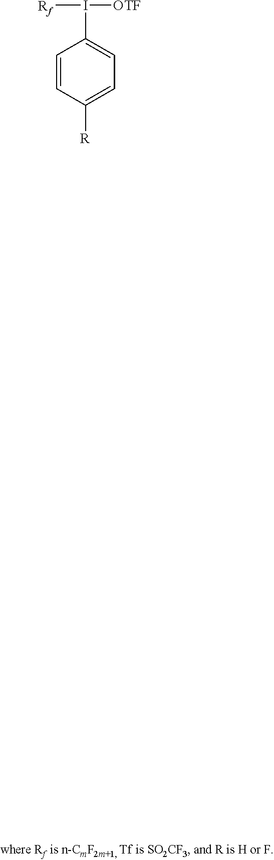

### SUMMARY

The technique of Japanese Unexamined Patent Application Publication No. 2018-43940 is not suitable for synthesis of a compound having a carbon-carbon double bond because an olefin is reacted, and the type of electrophile is limited.

In addition, since the product can further undergo a radical reaction to be telomerized, various types of by-products are generated.

In addition, the electrophilic perfluoroalkylating agent of Teruo Umemoto, “Electrophilic Perfluoroalkylating Agents”, Chem. Rev. 1996, 96, 1757-1777 as an electrophile requires a multi-step process for synthesis, has a low yield, and is expensive.

An object of the present invention is to provide a method for producing a fluorine-containing compound by using an easily available compound under relatively mild reaction conditions to produce a fluorine-containing compound, and a method for producing a surface treatment agent using the fluorine-containing compound obtained by the production method.

The present invention provides a method for producing a fluorine-containing compound and a method for producing a surface treatment agent having the following configurations [1] to [9].

[1] A method for producing a fluorine-containing compound represented by the following formula (C1) or (C2), the method including: reacting a compound represented by the following formula (A1) or (A2) with a compound represented by the following formula (B1) or (B2).

G¹-L¹-CR¹R²—X¹ Formula (A1)

X²—CR³R⁴-L²-G²-L³-CR⁵R⁶—X² Formula (A2)

R¹¹—ZnR¹² Formula (B1)

R¹¹—BR¹³R¹⁴ Formula (B2)

G¹-L¹-CR¹R²—R¹¹ Formula (C1)

R¹¹—CR³R⁴-L²-G²-L³-CR⁵R⁶—R¹¹ Formula (C2)

where, in the formula,
G¹ is a fluoroalkyl group or a monovalent group having a (poly)oxyfluoroalkylene chain,
G² is a fluoroalkylene group or a divalent group having a (poly)oxyfluoroalkylene chain,
L¹, L², and L³ are each independently a single bond or a divalent organic group,
R¹, R², R³, R⁴, R⁵, and R⁶ are each independently a hydrogen atom or an alkyl group having 1 to 6 carbon atoms which may have a substituent,
R¹¹ is a hydrocarbon group which may have a substituent or a heteroatom, and when there are a plurality of R¹¹'s, the R¹¹'s may be the same as or different from each other,
R¹² is a halogen atom or a hydrocarbon group which may have a substituent or a heteroatom,
R¹³ and R¹⁴ are each independently an alkyl group, an alkoxy group, and a hydroxyl group, or R¹³ and R¹⁴ are linked to form a ring structure, and X¹, X², and X³ are each independently a halogen atom.

[2] A method for producing a fluorine-containing compound represented by the following formula (C1) or (C2), the method including: reacting a compound represented by the following formula (A3) or (A4) with the following formula (B3).

G¹-L¹-CR¹R²—ZnR¹² Formula (A3)

R¹²Zn—CR³R⁴-L²-G²-L³-CR⁵R⁶—ZnR¹² Formula (A4)

R¹¹—X⁴ Formula (B3)

G¹-L¹-CR¹R²—R¹¹ Formula (C1)

R¹¹—CR³R⁴-L²-G²-L³-CR⁵R⁶—R¹¹ Formula (C2)

where, in the formula,
G¹ is a fluoroalkyl group or a monovalent group having a (poly)oxyfluoroalkylene chain,
G² is a fluoroalkylene group or a divalent group having a (poly)oxyfluoroalkylene chain,
L¹, L², and L³ are each independently a single bond or a divalent organic group,
R¹, R², R³, R⁴, R⁵, and R⁶ are each independently a hydrogen atom or an alkyl group having 1 to 6 carbon atoms which may have a substituent,
R¹¹ is a hydrocarbon group which may have a substituent or a heteroatom, and when there are a plurality of R¹¹'s, the R¹¹'s may be the same as or different from each other,
R¹² is a halogen atom or a hydrocarbon group which may have a substituent or a heteroatom, and when there are a plurality of R¹²'s, the R¹²'s may be the same as or different from each other, and
X⁴ is a halogen atom.

[3] The method for producing a fluorine-containing compound according to [1] or [2], in which at least one of the L¹-CR¹R², the L²-CR³R⁴, and the L³-CR⁵R⁶ is represented by (CR⁷R⁸—R⁹R¹⁰)ₙ₁,
where, in the formula,
R⁷, R⁸, R⁹, and R¹⁰ are each independently a hydrogen atom or an alkyl group having 1 to 6 carbon atoms which may have a substituent, and when there are a plurality of R⁷'s, R⁸'s, R⁹'s, and R¹⁰'s, the R⁷'s, R⁸'s, R⁹'s, and R¹⁰'s may be the same as or different from each other, and
n1 is an integer of 1 to 20.

[4] The method for producing a fluorine-containing compound according to any one of [1] to [3], in which at least one of the L¹-CR¹R², the L²-CR³R⁴, and the L³-CR⁵R⁶ is represented by (CH₂CH₂)ₙ₂, where, in the formula, n2 is an integer of 1 to 20.

[5] The method for producing a fluorine-containing compound according to any one of [1] to [4], in which the R¹¹ is represented by the following formula (D1).

(CH₂═CH—R²¹—)ₐ(R²²—)₃₋ₐC—R²³—* Formula (D1)

where, in the formula,
R²¹ is a single bond or an alkylene group which may have a fluorine atom having 1 to 18 carbon atoms, and when there are a plurality of R²¹'s, the R²¹'s may be the same as or different from each other,
R²² is a hydrogen atom or an alkyl group which may have a fluorine atom having 1 to 10 carbon atoms, and when there are a plurality of R²²'s, the R²²'s may be the same as or different from each other,
R²³ is a single bond or an alkylene group having 1 to 19 carbon atoms,
a is an integer of 1 to 3, and
* is an atomic bond.

[6] The method for producing a fluorine-containing compound according to any one of [1] to [5], in which at least one of the X¹, X², X³, and X⁴ is an iodine atom.

[7] The method for producing a fluorine-containing compound according to any one of [1] to [6], in which the reaction is performed in the presence of a transition metal compound.

[8] The method for producing a fluorine-containing compound according to [7], in which the transition metal compound contains one or more elements selected from Ni and Pd.

[9] A method for producing a surface treatment agent, including: producing a fluorine-containing compound represented by the formula (C1) or (C2) by the production method according to any one of [1] to [8]; and introducing a reactive silyl group into the fluorine-containing compound.

The above and other objects, features and advantages of the present disclosure will become more fully understood from the detailed description given hereinbelow which are given by way of illustration only, and thus are not to be considered as limiting the present disclosure.

According to the present invention, it is possible to provide a method for producing a fluorine-containing compound by using an easily available compound under relatively mild reaction conditions to produce a fluorine-containing compound, and a method for producing a surface treatment agent using the fluorine-containing compound obtained by the production method.

### DESCRIPTION OF EMBODIMENTS

In the present specification, the compound represented by the formula (A1) is referred to as a compound (A1). The same applies to compounds represented by other formulae and the like.

The “(poly)oxyfluoroalkylene” is a generic term for oxyfluoroalkylene and polyoxyfluoroalkylene.

The fluoroalkyl group is a generic term for a combination of a perfluoroalkyl group and a partial fluoroalkyl group. The perfluoroalkyl group means a group in which all hydrogen atoms of the alkyl group are substituted with fluorine atoms. In addition, the partial fluoroalkyl group is an alkyl group in which one or more hydrogen atoms are substituted with a fluorine atom and which has one or more hydrogen atoms. That is, the fluoroalkyl group is an alkyl group having one or more fluorine atoms.

The “reactive silyl group” is a generic term for a hydrolyzable silyl group and a silanol group (Si—OH), and the “hydrolyzable silyl group” means a group capable of forming a silanol group by a hydrolysis reaction.

The “organic group” means a hydrocarbon group which may have a substituent or a heteroatom or other bonds in a carbon chain. The “hydrocarbon group” is a group including an aliphatic hydrocarbon group (linear alkylene group, branched alkylene group, cycloalkylene group, and the like), an aromatic hydrocarbon group (phenylene group and the like), and a combination thereof.

The “surface layer” means a layer formed on the surface of the substrate.

“to” indicating a numerical range means that the numerical values stated before and after “to” are included as a lower limit value and an upper limit value.

### [Method for Producing Fluorine-Containing Compound]

The method for producing a fluorine-containing compound of the present invention (hereinafter also referred to as the present production method) is a preferred production method capable of introducing any substituent into a compound having a fluoroalkylene chain or a (poly)oxyfluoroalkylene chain by subjecting an organohalogen compound or an organozinc compound having a fluoroalkylene chain or a (poly)oxyfluoroalkylene chain, and an organozinc compound, an organoboron compound or an organohalogen compound having any substituent to a coupling reaction. According to the present production method, even a compound having a relatively high molecular weight (long chain) (poly)oxyfluoroalkylene chain has high reaction efficiency, and the yield of a target product can be increased while suppressing the reaction temperature and reaction time. For example, according to the present production method, even a compound having a (poly)oxyfluoroalkylene chain having a molecular weight of 200 to 30,000 can be suitably produced as a target product. In addition, according to the present production method, for example, a fluorine-containing compound having a molecular weight of 1,000 to 30,000 can be suitably produced.

Hereinafter, the production methods belonging to the present production method will be described in more detail.

### <First Production Method>

A first production method of a fluorine-containing compound of the present invention is a method for producing a fluorine-containing compound represented by the following formula (C1) or (C2), the method including: reacting a compound represented by the following formula (A1) or (A2) with a compound represented by the following formula (B1) or (B2).

X²—CR³R⁴-L²-G²-L³-CR⁵R⁶—X² Formula (A2)

R¹¹—ZnR¹² Formula (B1)

R¹¹—BR¹³R¹⁴ Formula (B2)

G¹-L¹-CR¹R²—R¹¹ Formula (C1)

R¹¹—CR³R⁴-L²-G²-L³-CR⁵R⁶—R¹¹ Formula (C2)

where, in the formula,
G¹ is a fluoroalkyl group or a monovalent group having a (poly)oxyfluoroalkylene chain,
G² is a fluoroalkylene group or a divalent group having a (poly)oxyfluoroalkylene chain,
L¹, L², and L³ are each independently a single bond or a divalent organic group,
R¹, R², R³, R⁴, R⁵, and R⁶ are each independently a hydrogen atom or an alkyl group having 1 to 6 carbon atoms which may have a substituent,
R¹¹ is a hydrocarbon group which may have a substituent or a heteroatom, and when there are a plurality of R¹¹'s, the R¹¹'s may be the same as or different from each other,
R¹² is a halogen atom or a hydrocarbon group which may have a substituent or a heteroatom,
R¹³ and R¹⁴ are each independently an alkyl group, an alkoxy group, and a hydroxyl group, or R¹³ and R¹⁴ are linked to form a ring structure, and
X¹, X², and X³ are each independently a halogen atom.

The first production method is a method of synthesizing the compound (C1) or the compound (C2) by subjecting the organohalogen compound (A1) or the compound (A2) having a fluoroalkyl chain or a (poly)oxyfluoroalkylene chain and the organozinc compound (B1) or the organoboron compound (B2) having a substituent R¹¹ to a coupling reaction.

The fluoroalkyl group in G¹ may be a linear fluoroalkyl group or a fluoroalkyl group having a branched or ring structure. The number of carbon atoms in the fluoroalkyl group is preferably 1 to 30, more preferably 1 to 20, still more preferably 1 to 10, and particularly preferably 1 to 6 from the viewpoint of increasing the yield of the present production method.

Specific examples of the fluoroalkyl group include CF₃—, CHF₂—, CF₃CF₂—, CF₃CHF—, CF₃CF₂CF₂—, CF₃CHFCF₂—, CF₃CHFCHF—, CF₃CF(CF₃)—, CF₃CF₂CF₂CF₂—, CF₃CHFCF₂CF₂—, CF₃CF(CF₃)CF₂—, CF₃C(CF₃)₂CF₂—, CF₃CF₂CF₂CF₂CF₂—, CF₃CF₂CF₂CF₂CF₂CF₂—, a fluorocyclobutyl group, a fluorocyclopentyl group, and a fluorocyclohexyl group.

The monovalent group having a (poly)oxyfluoroalkylene chain in G¹ is a fluoroalkyl group having —O— at a terminal bonded to L¹ (when L¹ is a single bond, CR¹R²), —O— between carbon-carbon atoms of a carbon chain having 2 or more carbon atoms, or both of them. From the viewpoint of ease of production and the like, G¹ preferably has a structure represented by the following formula (G¹-1).

R^{f0}O—[(R^{f1}O)ₘ₁(R^{f2}O)ₘ₂(R^{f3}O)ₘ₃(R^{f4}O)ₘ₄(R^{f5}O)ₘ₅(R^{f6}O)ₘ₆]—(R^{f7})ₘ₇— Formula (G1-1)

where
R^{f0} is a fluoroalkyl group having 1 to 20 carbon atoms,
R^{f1} is a fluoroalkylene group having 1 carbon atom,
R^{f2} is a fluoroalkylene group having 2 carbon atoms,
R^{f3} is a fluoroalkylene group having 3 carbon atoms,
R^{f4} is a fluoroalkylene group having 4 carbon atoms,
R^{f5} is a fluoroalkylene group having 5 carbon atoms,
R^{f6} is a fluoroalkylene group having 6 carbon atoms,
R^{f7} is a fluoroalkylene group having 1 to 6 carbon atoms,
m1, m2, m3, m4, m5, and m6 each independently represent an integer of 0 or 1 or more, m7 is an integer of 0 or 1, and m1+m2+m3+m4+m5+m6 is an integer of 1 to 200. When the obtained compound (C1) is used as a surface treatment agent or a raw material thereof, m1+m2+m3+m4+m5+m6 is an integer of 1 to 200, that is, G¹ is preferably a polyoxyfluoroalkylene chain from the viewpoint of water/oil repellency, fingerprint removability, and the like.

Note that the bonding order of (R^{f1}O) to (R^{f6}O) in the formula (G1-1) is random.

m1 to m6 in the formula (G1-1) represent the number of (R^{f1}O) to (R^{f6}O), respectively, and do not represent arrangement. For example, (R⁵)ₘ₅ represents that the number of (R^{f5}O) is m5, and does not represent a block arrangement structure of (R⁵)ₘ₅. Similarly, the order of description of (R^{f1}O) to (R^{f6}O) does not represent the bonding order of the respective units.

When m7 is 0, the terminal of G¹ bonded to L¹ (when L¹ is a single bond, CR¹R²) is —O—. When m7 is 1, the terminal of G¹ bonded to L¹ (when L¹ is a single bond, CR¹R²) is a carbon atom (carbon atom at the terminal of R^{f7}).

In addition, the fluoroalkylene group having 3 to 6 carbon atoms may be a linear fluoroalkylene group or a fluoroalkylene group having a branched or ring structure.

Specific examples of R^{f1} include —CF₂— and —CHF—.

Specific examples of R^{f2} include —CF₂CF₂—, —CHFCF₂—, —CHFCHF—, —CH₂CF₂—, and —CH₂CHF—.

Specific examples of R^{f3} include —CF₂CF₂CF₂—, —CF₂CHFCF₂—, —CF₂CH₂CF₂—, —CHFCF₂CF₂—, —CHFCHFCF₂—, —CHFCHFCHF—, —CHFCH₂CF₂—, —CH₂CF₂CF₂—, —CH₂CHFCF₂—, —CH₂CH₂CF₂—, —CH₂CF₂CHF—, —CH₂CHFCHF—, —CH₂CH₂CHF—, —CF(CF₃)—CF₂—, —CF(CHF₂)—CF₂—, —CF(CH₂F)—CF₂—, —CF(CH₃)—CF₂—, —CF(CF₃)—CHF—, —CF(CHF₂)—CHF—, —CF(CH₂F)—CHF—, —CF(CH₃)—CHF—, —CF(CF₃)—CH₂—, —CF(CHF₂)—CH₂—, —CF(CH₂F)—CH₂—, —CF(CH₃)—CH₂—, —CH(CF₃)—CF₂—, —CH(CHF₂)—CF₂—, —CH(CH₂F)—CF₂—, —CH(CH₃)—CF₂—, —CH(CF₃)—CHF—, —CH(CHF₂)—CHF—, —CH(CH₂F)—CHF—, —CH(CH₃)—CHF—, —CH(CF₃)—CH₂—, —CH(CHF₂)—CH₂—, and —CH(CH₂F)—CH₂—.

Specific examples of R^{f4} include —CF₂CF₂CF₂CF₂—, —CHFCF₂CF₂CF₂—, —CH₂CF₂CF₂CF₂—, —CF₂CHFCF₂CF₂—, —CHFCHFCF₂CF₂—, —CH₂CHFCF₂CF₂—, —CF₂CH₂CF₂CF₂—, —CHFCH₂CF₂CF₂—, —CH₂CH₂CF₂CF₂—, —CHFCF₂CHFCF₂—, —CH₂CF₂CHFCF₂—, —CF₂CHFCHFCF₂—, —CHFCHFCHFCF₂—, —CH₂CHFCHFCF₂—, —CF₂CH₂CHFCF₂—, —CHFCH₂CHFCF₂—, —CH₂CH₂CHFCF₂—, —CF₂CH₂CH₂CF₂—, —CHFCH₂CH₂CF₂—, —CH₂CH₂CH₂CF₂—, —CHFCH₂CH₂CHF—, —CH₂CH₂CH₂CHF—, and -cycloC₄F₆—.

Specific examples of R^{f5} include —CF₂CF₂CF₂CF₂CF₂—, —CHFCF₂CF₂CF₂CF₂—, —CH₂CHFCF₂CF₂CF₂—, —CF₂CHFCF₂CF₂CF₂—, —CHFCHFCF₂CF₂CF₂—, —CF₂CH₂CF₂CF₂CF₂—, —CHFCH₂CF₂CF₂CF₂—, —CH₂CH₂CF₂CF₂CF₂—, —CF₂CF₂CHFCF₂CF₂—, —CHFCF₂CHFCF₂CF₂—, —CH₂CF₂CHFCF₂CF₂—, —CH₂CF₂CF₂CF₂CH₂—, and -cycloC₅F₈—.

Specific examples of R^{f6} include —CF₂CF₂CF₂CF₂CF₂CF₂—, —CF₂CF₂CHFCHFCF₂CF₂—, —CHFCF₂CF₂CF₂CF₂CF₂—, —CHFCHFCHFCHFCHFCHF—, —CHFCF₂CF₂CF₂CF₂CH₂—, —CH₂CF₂CF₂CF₂CF₂CH₂—, and -cycloC₆F₁₀—.

In addition, specific examples of R^{f0} and R^{f7} include the same ones as those mentioned in the above R^{f1} to R^{f6}.

Here, -cycloC₄F₆— means a perfluorocyclobutanediyl group, and specific examples thereof include a perfluorocyclobutane-1,2-diyl group. -cycloC₅F₈-means a perfluorocyclopentanediyl group, and specific examples thereof include a perfluorocyclopentane-1,3-diyl group. -cycloC₆F₁₀— means a perfluorocyclohexanediyl group, and specific examples thereof include a perfluorocyclohexane-1,4-diyl group.

When the obtained compound (C1) is used as a surface treatment agent or a raw material thereof, G¹ is preferably a monovalent group having a (poly)oxyfluoroalkylene chain, and more preferably a monovalent group having a polyoxyfluoroalkylene chain, from the viewpoint of further excellent water/oil repellency, friction resistance, and fingerprint dirt removability. Among them, it is preferable to have structures represented by the following formulae (F2) to (F4).

(R^{f1}O)ₘ₁—(R^{f2}O)ₘ₂ Formula (F1)

(R^{f2}O)ₘ₂—(R^{f4}O)ₘ₄ Formula (F2)

(R^{f3}O)ₘ₃ Formula (F3)

where, each reference sign of the formulae (F1) to (F3) is the same as those in the formula (G1-1).

In the formulae (F1) and (F2), the bonding order of (R^{f1}O) and (R^{f2}O), and (R^{f2}O) and (R^{f4}O) is random. For example, (R^{f1}O) and (R^{f2}O) may be alternately arranged, (R^{f1}O) and (R^{f2}O) may be each arranged in a block, or may be random.

The same applies to the formula (F3).

In the formula (F1), m1 is preferably 1 to 50, more preferably 1 to 40, still more preferably 1 to 30, and particularly preferably 1 to 20. In addition, m2 is preferably 1 to 50, more preferably 1 to 40, still more preferably 1 to 30, and particularly preferably 1 to 20.

In the formula (F2), m2 is preferably 1 to 50, more preferably 1 to 40, still more preferably 1 to 30, and particularly preferably 1 to 20. In addition, m4 is preferably 1 to 50, more preferably 1 to 40, still more preferably 1 to 30, and particularly preferably 1 to 20.

In the formula (F3), m3 is preferably 1 to 50, more preferably 1 to 40, still more preferably 1 to 30, and particularly preferably 1 to 20.

The fluoroalkylene group in G² may be linear, or may have a branched or ring structure. The number of carbon atoms in the fluoroalkylene group is preferably 1 to 30, more preferably 1 to 20, still more preferably 1 to 10, and particularly preferably 1 to 6 from the viewpoint of increasing the yield of the present production method.

Specific examples of the fluoroalkylene group include the same groups as those mentioned in the above R^{f1} to R^{f6}.

The divalent group having a (poly)oxyfluoroalkylene chain in G² is a fluoroalkylene group having —O— at two terminals bonded to L² or L³ (when L² or L³ is a single bond, CR³R⁴ or CR⁵R⁶) each independently, or —O— between carbon-carbon atoms of a carbon chain having 2 or more carbon atoms, or is a combination thereof in the formula (A2). From the viewpoint of ease of production and the like, G² preferably has a structure represented by the following formula (G2-1).

—(O)ₘ₀—[(R^{f1}O)ₘ₁(R^{f2}O)ₘ₂(R^{f3}O)ₘ₃(R^{f4}O)ₘ₄(R^{f5}O)ₘ₅(R^{f6}O)ₘ₆]—(R^{f7})ₘ₇— Formula (G2-1)

Here, m0 is an integer of 0 or 1, and R^{f1}, R^{f2}, R^{f3}, R^{f4}, R^{f5}, R^{f6}, R^{f7}, m1, m2, m3, m4, m5, m6, and m7 are the same as those in the G¹. Note that the bonding order of (R^{f1}O) to (R^{f6}O) in the formula (G2-1) is random and is as described in the above formula (G1-1).

When m7 is 0, one terminal of G² bonded to L³ (when L³ is a single bond, CR⁵R⁶) is —O—. When m7 is 1, one terminal of G² bonded to L³ (when L³ is a single bond, CR⁵R⁶) is a carbon atom (carbon atom at the terminal of R^{f7}). In addition, when m0 is 1, one terminal of G² bonded to L² (when L² is a single bond, CR³R⁴) is —O—. When m0 is 0, one terminal of G² bonded to L² (when L² is a single bond, CR³R⁴) is a carbon atom (carbon atom at any terminal of R^{f1} to R^{f7}). Note that m0 and m7 are each independently 0 or 1.

When the obtained compound (C2) is used as a surface treatment agent or a raw material thereof, m1+m2+m3+m4+m5+m6 is an integer of 1 to 200, that is, G² is preferably a polyoxyfluoroalkylene chain from the viewpoint of water/oil repellency, fingerprint removability, and the like.

When the obtained compound (C2) is used as a surface treatment agent or a raw material thereof, G² preferably has a structure represented by the following formulae (F4) to (F6) from the viewpoint of further excellent water/oil repellency, friction resistance, and fingerprint dirt removability.

—(O)ₘ₀—(R^{f1}O)ₘ₁—(R^{f2}O)ₘ₂ Formula (F4)

—(O)ₘ₀—(R^{f2}O)ₘ₂—(R^{f4}O)ₘ₄ Formula (F5)

—(O)ₘ₀—(R^{f3}O)ₘ₃ Formula (F6)

where, each reference sign of the formulae (F4) to (F6) is the same as those in the formula (G2-1).

In the formulae (F4) and (F5), the bonding order of (R^{f1}O) and (R^{f2}O), and (R^{f2}O) and (R^{f4}O) is random. For example, (R^{f1}O) and (R^{f2}O) may be alternately arranged, (R^{f1}O) and (R^{f2}O) may be each arranged in a block, or may be random. The same applies to the formula (F6).

In the formula (F4), m1 is preferably 1 to 50, more preferably 1 to 40, still more preferably 1 to 30, and particularly preferably 1 to 20. In addition, m2 is preferably 1 to 50, more preferably 1 to 40, still more preferably 1 to 30, and particularly preferably 1 to 20.

In the formula (F5), m2 is preferably 1 to 50, more preferably 1 to 40, still more preferably 1 to 30, and particularly preferably 1 to 20. In addition, m4 is preferably 1 to 50, more preferably 1 to 40, still more preferably 1 to 30, and particularly preferably 1 to 20.

In the formula (F6), m3 is preferably 1 to 50, more preferably 1 to 40, still more preferably 1 to 30, and particularly preferably 1 to 20.

The ratio of fluorine atoms in the fluoroalkyl chain and the (poly)oxyfluoroalkylene chain [{number of fluorine atoms/(number of fluorine atoms+number of hydrogen atoms)}×100(%)] in G¹ and G² is preferably 40% or more, more preferably 50% or more, and still more preferably 60% or more from the viewpoint of excellent water/oil repellency and fingerprint removability.

In addition, the molecular weight of the (poly)oxyfluoroalkylene chain part is preferably 200 to 30,000, more preferably 600 to 25,000, and still more preferably 1,000 to 20,000 from the viewpoint of wear resistance.

L¹, L², and L³ are each independently a single bond or a divalent organic group. Examples of the organic group in L¹, L², and L³ include a hydrocarbon group which may have a substituent or a heteroatom or other bonds (B¹) in the carbon chain.

Examples of the hydrocarbon group include an aliphatic hydrocarbon group (linear alkylene group, branched alkylene group, cycloalkylene group, and the like), an aromatic hydrocarbon group (phenylene group and the like), and a group consisting of combination thereof. The aliphatic hydrocarbon group may have a double bond or a triple bond in the carbon chain. Examples of the combination include a group in which an alkylene group and an arylene group are directly linked via a heteroatom or other bonds.

Examples of the substituent that the hydrocarbon group may have include a halogen atom, a hydroxy group, an amino group, a nitro group, and a sulfo group, and from the viewpoint of the stability of the compound in the present production method, a halogen atom is preferable. Examples of the halogen atom include a fluorine atom, a chlorine atom, and a bromine atom.

Specific examples of the heteroatom or other bonds (B¹) include —C(O)NR²⁶—, —C(O)O—, —C(O)—, —O—, —NR²⁶—, —S—, —OC(O)O—, —NHC(O)O—, —NHC(O)NR²⁶—, —SO₂NR²⁶—, —Si(R²⁶)₂—, —OSi(R²⁶)₂—, —Si(CH₃)₂-Ph-Si(CH₃)₂—, and a divalent organopolysiloxane residue. Here, R²⁶ is a hydrogen atom, an alkyl group having 1 to 6 carbon atoms, or a phenyl group, and Ph is a phenylene group. From the viewpoint of ease of production of the present compound, the number of carbon atoms in the alkyl group of R²⁶ is preferably 1 to 3, and particularly preferably 1 and 2.

Specific examples of L¹, L², and L³ include a single bond, an alkylene group R²⁸ which may have a substituent, and a combination of an alkylene group R²⁸ which may have a substituent and the B¹ (for example, —R²⁸—B¹—, —B¹—R²⁸—B¹—, and —R²⁸—B¹—R²⁸—).

R¹, R², R³, R⁴, R⁵, and R⁶ are each independently a hydrogen atom or an alkyl group having 1 to 6 carbon atoms which may have a substituent. Examples of the alkyl group include a linear or branched alkyl group. Examples of the substituent that the alkyl group may have include a halogen atom, a hydroxy group, an amino group, a nitro group, and a sulfo group, and from the viewpoint of the stability of the compound in the present production method, a halogen atom is preferable. Examples of the halogen atom include a fluorine atom, a chlorine atom, and a bromine atom. Among them, the halogen atom is preferably a fluorine atom from the viewpoint of stability. Specific examples of the alkyl group which may have a substituent include CH₃—, CH₂F—, CHF₂—, CF₃—, CH₃CH₂—, CF₃CH₂—, CF₃CF₂—, CH₃CH₂CH₂—, CF₃CH₂CH₂—, CF₃CF₂CH₂—, CF₃CF₂CF₂—, CH₃CH(CH₃)—, CF₃CH(CH₃)—, CF₃CH(CF₃)—, CF₃CF(CF₃)—, CH₃CH₂CH₂CH₂—, CF₃CF₂CF₂CF₂—, CH₃CH₂CH(—CH₂CH₃)—, CF₃CF₂CF(—CF₂CF₃)—, CH₃CH₂CH₂CH(—CH₂CH₃)—, and CF₃CF₂CF₂CF(—CF₂CF₃)—. In addition, the alkyl groups which may have substituents of R¹, R², R³, R⁴, R⁵, and R⁶ may be the same as or different from each other.

R¹, R², R³, R⁴, R⁵, and R⁶ are preferably a hydrogen atom from the viewpoint of reactivity.

In the present production method, at least one of L¹-CR¹R², L²-CR³R⁴, and L³-CR⁵R⁶ in the compound (A1) or the compound (A2) preferably has a structure represented by (CR⁷R⁸—CR⁹R¹⁰)ₙ₁ from the viewpoint of ease of synthesis of raw materials, reactivity with the compound (A1), the compound (A2), and the compound (B2), and the like.

where, in the formula,
R⁷, R¹, R⁹, and R¹⁰ are each independently a hydrogen atom or an alkyl group having 1 to 6 carbon atoms which may have a substituent, and when there are a plurality of R⁷'s, R⁸'s, R⁹'s, and R¹⁰'s, the R⁷'s, R⁸'s, R⁹'s, and R¹⁰'s may be the same as or different from each other, and
n1 is an integer of 1 to 20.

For example, when L¹-CR¹R² is (CR⁷R⁸—CR⁹R¹⁰)ₙ₁, the compound (A1) is represented by the following formula (Ala). In addition, for example, when L²-CR³R⁴ and L³-CR⁵R⁶ are (CR⁷R⁸—CR⁹R¹⁰)ₙ₁, the compound (A2) is represented by the following formula (A2a). Further, the compound (A3), the compound (A4), the compound (C1), the compound (C2), and the like, which will be described later, also follow this.

G¹-(CR⁷R⁸—CR⁹R¹⁰)ₙ₁—X¹ Formula (A1a)

X²—(CR⁹R¹⁰—CR⁷R⁸)ₙ₁-L²-G²-(CR⁷R⁸—CR⁹R¹⁰)ₙ₁—X³ Formula (A2a)

The alkyl group in R⁷, R⁸, R⁹, and R¹⁰ is the same as that in R¹ to R⁶. Among them, from the viewpoint of reactivity, it is preferable that R⁹ and R¹⁰ be a hydrogen atom, that is, (CR⁷R⁸—CR⁹R¹⁰)ₙ₁ is a group represented by (CR⁷R⁸—CH₂)ₙ₁. In addition, in (CR⁷R⁸—CH₂)ₙ₁, the atomic bond of “CH₂” is bonded to X¹, X², or X³ of the compound (A1) or the compound (A2). When the compound (A1) and the compound (A2) have the structure of “—CH₂—X¹¹” (where X¹¹ is X¹, X², or X³), the reactivity of coupling in the present production method is improved.

Examples of the halogen atom in X¹, X², and X³ include a fluorine atom, a chlorine atom, a bromine atom, and an iodine atom, and an iodine atom is preferable from the viewpoint of further improving the reactivity.

Specific examples of (CR⁷R⁸—CH₂)ₙ₁ include CH₂CH₂, CH₂CH₂CH₂CH₂, CH₂CH₂CH₂CH₂CH₂CH₂, CH(—CH₃)CH₂, CH(—CF₃)CH₂, CH(—CH₂F)CH₂, CH(—CHF₂)CH₂, C(—CH₃)(—CH₃)CH₂, C(—CF₃)(—CF₃)CH₂, C(—CH₂CH₃)(—CH₂CH₃)CH₂, C(—CF₂CF₃)(—CF₂CF₃)CH₂, C(—CH₂CH₂CH₃)(—CH₂CH₂CH₃)CH₂, C(—CF₂CF₂CF₃)(—CF₂CF₂CF₃)CH₂, C(—CH₂CH₂CH₂CH₃)(—CH₂CH₂CH₂CH₃)CH₂, C(—CF₂CF₂CF₂CF₃)(—CF₂CF₂CF₂CF₃)CH₂, C(—CH₂CH₂CH₂CH₂CH₃)(—CH₂CH₂CH₂CH₂CH₃)CH₂, C(—CF₂CF₂CF₂CF₂CF₃)(—CF₂CF₂CF₂CF₂CF₃)CH₂, C(—CH₂CH₂CH₂CH₂CH₂CH₃)(—CH₂CH₂CH₂CH₂CH₂CH₃)CH₂, and C(—CF₂CF₂CF₂CF₂CF₂CF₃)(—CF₂CF₂CF₂CF₂CF₂CF₃)CH₂.

Furthermore, in the present production method, particularly R⁷ is a hydrogen atom, R⁸ is preferably a hydrogen atom or a methyl group, and both R⁷ and R⁸ are more preferably a hydrogen atom, from the viewpoint of ease of synthesis of raw materials, reactivity with the compound (A1), the compound (A2), and the compound (B2), and the like. That is, at least one of L¹-CR¹R², L²-CR³R⁴, and L³-CR⁵R⁶ in the compound (A1) or the compound (A2) preferably has a structure represented by (CH₂CH₂)ₙ₂. However, n2 is an integer of 1 to 20, preferably 1 to 12, and more preferably 1 to 6.

Preferred specific examples of the compound (A1) and the compound (A2) include the following compounds.

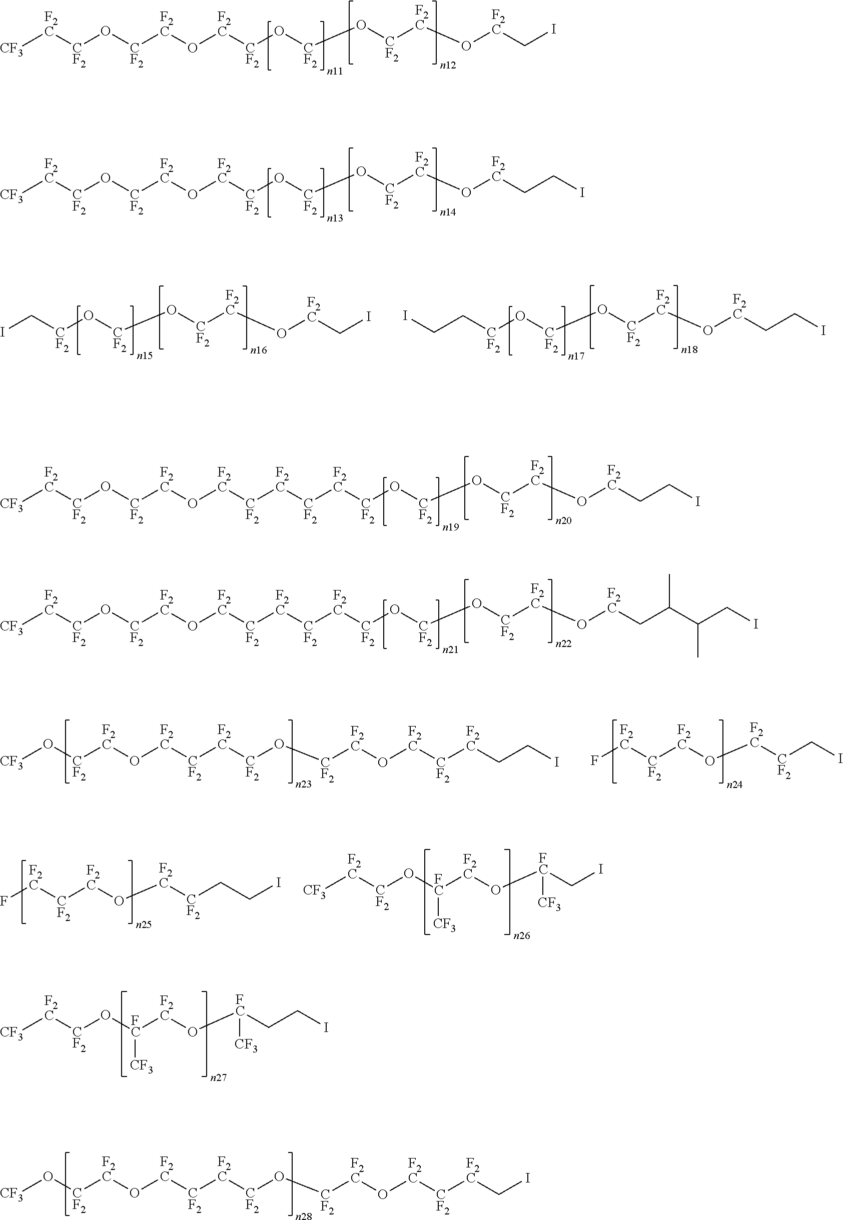

where, n11 to n28 represent the number of repeating units, and each independently represent an integer of 1 to 200.

The compound (A1) and the compound (A2) can be produced, for example, by a method of reacting the compound represented by the following formulae (A1-2) and (A2-2) with triphenylphosphine and iodomethane to iodinate the compound, a method of reacting the compound with triphenylphosphine and iodine to iodinate the compound, or the like. In addition, a commercially available product having a desired structure may be used.

G¹-L¹-CR¹R²—OH Formula (A1-2)

HO—CR³R⁴-L²-G²-L³-CR⁵R⁶—OH Formula (A2-2)

where, each reference sign in the formula is as described above.

In addition, as an example of synthesis of the compound (A1), a compound represented by the following formula (A1-3) or the like can also be produced by adding an initiator, a metal catalyst, an organic catalyst or the like, and ethylene to the following formula (A1-4) and reacting them.

G¹-L¹-CH₂CH₂—X¹ Formula (A1-3)

G¹-L¹-X¹ Formula (A1-4)

In addition, the initiator, the metal catalyst, and the organic catalyst can be appropriately selected from known initiators and used. Examples of the initiator include an azo-based initiator, an organic peroxide, and a redox initiator.

Examples of the metal catalyst include simple metals such as copper and iron, and copper acetate, and copper chloride. In addition, examples of the organic catalyst include triethoxyphosphine.

Further, in order to obtain the compound (A1) having a desired structure, another olefin compound may be used instead of ethylene.

In the compounds (B1) and (B2), R¹¹ is a substituent to be introduced into the compound (A1) and the compound (A2), and can be appropriately selected and used according to the application of the compound (C1) and the compound (C2) to be obtained, and the like.

Examples of the hydrocarbon group in R¹¹ include a hydrocarbon group which may have a substituent or a heteroatom or other bonds (B1) in the carbon chain.

Examples of the hydrocarbon group include an aliphatic hydrocarbon group (linear alkyl group, branched alkyl group, cycloalkyl group, and the like), an aromatic hydrocarbon group (phenyl group and the like), and a group consisting of combinations thereof. The aliphatic hydrocarbon group may have a double bond or a triple bond in the carbon chain. Examples of the combination include a group in which an alkylene group and an aryl group are directly linked via a heteroatom or other bonds.

Examples of the substituent that the hydrocarbon group may have include a halogen atom, a hydroxy group, an amino group, a nitro group, and a sulfo group, and from the viewpoint of the stability of the compound in the present production method, a halogen atom is preferable. Examples of the halogen atom include a fluorine atom, a chlorine atom, and a bromine atom.

Specific examples of the heteroatom or other bonds include the same ones as those mentioned above for B¹.

When another substituent is further introduced using the obtained compound (C1) or compound (C2) as a raw material, as an example, R¹¹ is preferably an alkyl group having a double bond. When R¹¹ is an alkyl group having a double bond, it is possible to suitably obtain the compound (C1) or compound (C2) into which a double bond is introduced, into which another substituent is easily introduced, while suppressing side reactions in the present production method.

Among them, a substituent represented by the following formula (D1) is preferable as R¹¹.

(CH₂═CH—R²¹—)ₐ(R²²—)₃₋ₐC—R²³—* Formula (D1)

where, in the formula,
R²¹ is a single bond or an alkylene group which may have a fluorine atom having 1 to 18 carbon atoms, and when there are a plurality of R²¹'s, the R²¹'s may be the same as or different from each other,
R²² is a hydrogen atom or an alkyl group which may have a fluorine atom having 1 to 10 carbon atoms, and when there are a plurality of R²²'s, the R²²'s may be the same as or different from each other,
R²³ is a single bond or an alkylene group having 1 to 19 carbon atoms,
a is an integer of 1 to 3, and
* is an atomic bond.

The group represented by (CH₂═CH—R²¹—) may contain an isomerized structure. For example, when the group represented by (CH₂═CH—R²¹—) is a group represented by CH₂═CH—CH₂—, a group represented by CH₃CH═CH— may be contained.

In addition, when a of the group represented by (CH₂═CH—R²¹—)ₐ is 2 or more, each of the groups may be the same as or different from each other.

The number of carbon atoms of R²¹ may be 1 to 18, and is preferably 1 to 8.

R¹² is a halogen atom or a hydrocarbon group which may have a substituent or a heteroatom.

From the viewpoint of reactivity, the halogen atom in R¹² is preferably a chlorine atom, a bromine atom, or an iodine atom, and more preferably a chlorine atom or a bromine atom.

Examples of the hydrocarbon group in R¹² include those similar to those in the R¹¹. In addition, when R¹² is a hydrocarbon group, R¹² may be introduced instead of R¹¹ in the reaction in the present production method, and for example, the following compounds (C3) to (C6) and the like may be generated.

G¹-L¹-CR¹R²—R¹² Formula (C3)

R¹¹—CR³R⁴-L²-G²-L³-CR⁵R⁶—R¹² Formula (C4)

R¹²—CR³R⁴-L²-G²-L³-CR⁵R⁶-R¹² Formula (C5)

R¹¹-R¹² Formula (C6)

where, each reference sign in the formula is as described above.

In this regard, the following correspondence can be made.

By having R¹¹ and R¹² as substituents having the same structure, the compounds (C3) to (C5) as by-products are the same compounds as the compound (C1) or (C2).

When R¹² is a substituent lower in reactivity than R¹¹, generation of the compounds (C3) to (C5) as by-products can be suppressed. For example, when R¹¹— is a substituent represented by R³¹—CH₂— (where R³¹ is a hydrocarbon group) and R¹²— is a substituent represented by R³¹—CR³²R³³— (where R³¹ is a hydrocarbon group, R³² and R³³ are each independently a hydrogen atom or an alkyl group, and at least one of them is an alkyl group), R¹¹ is preferentially introduced in the reaction by the present production method.

In addition, when the compounds (C3) to (C6) are produced, separation by column chromatography or the like may be performed as necessary, and a mixture containing the compounds (C3) to (C6) may be used as it is depending on the application of the compound (C1) or (C2).

These may be appropriately selected depending on the application of the compound (C1) or the compound (C2).

Preferred specific examples of the compound (B1) include the following

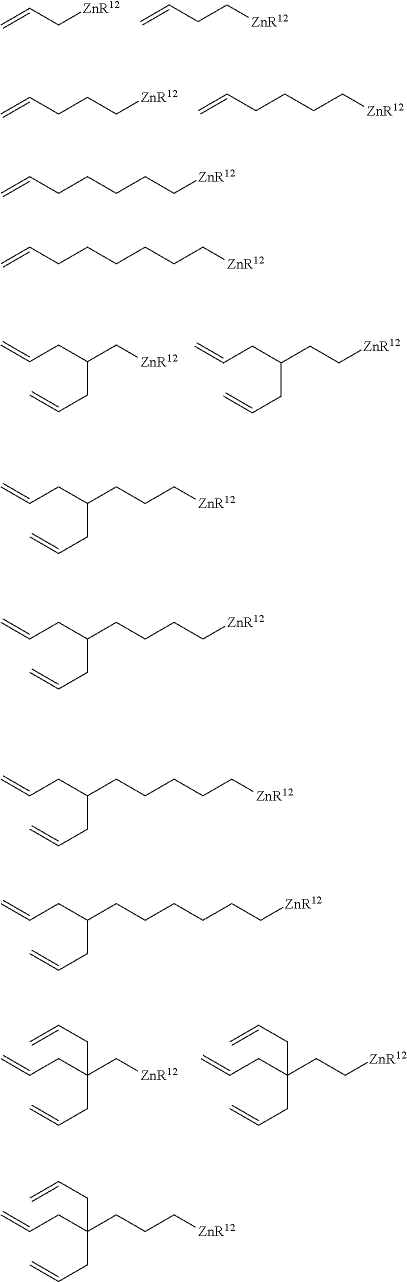

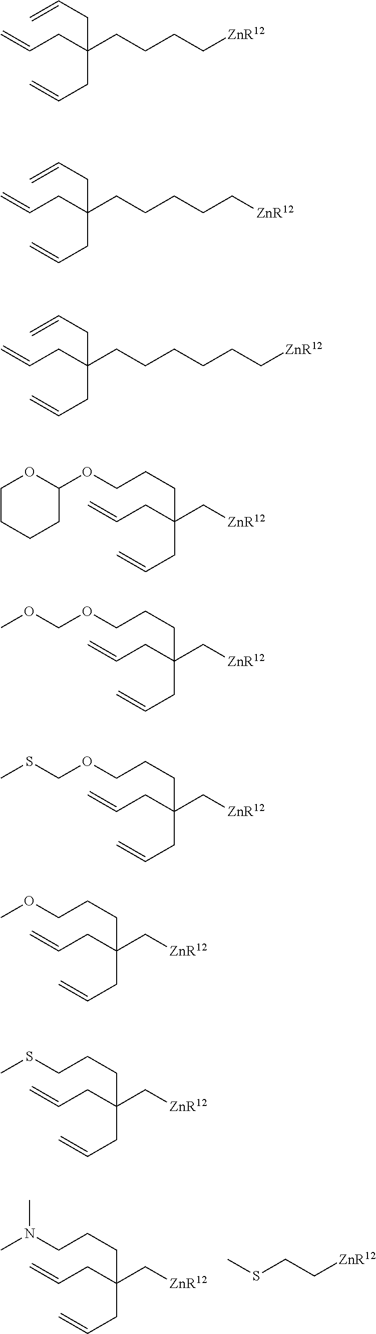

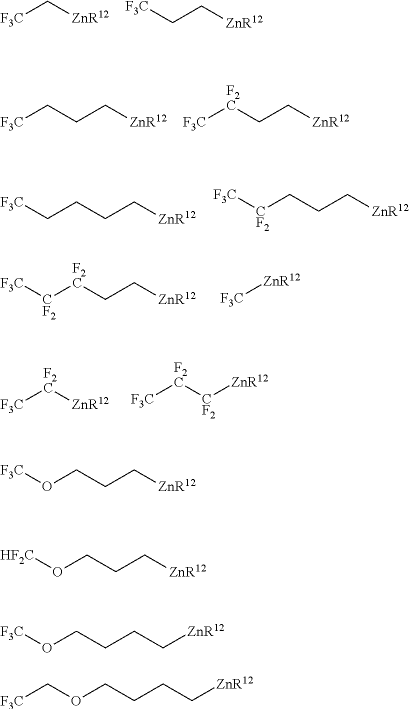

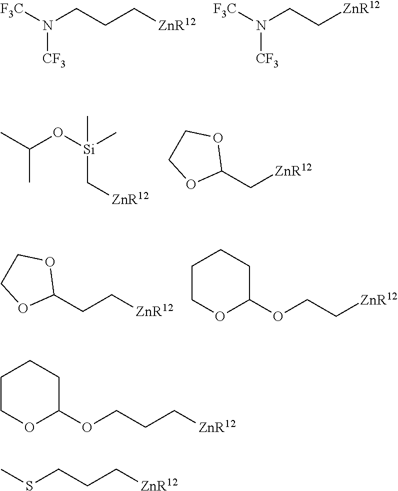

where, R¹² is as described above.

Regarding the compound (B1), for example, an organozinc compound in which R¹² is a halogen atom is obtained by reacting a compound represented by the following formula (B1-1) with Rieke zinc or the like. In addition, by reacting with a compound represented by the following formula (B1-2), the compound (B1) in which R¹¹ and R¹² have the same structure is obtained.

R¹¹—X⁵ Formula (B1-1)

R¹¹—ZnX⁵ Formula (B1-2)

where, R¹¹ is as described above, and X⁵ is a halogen atom.

The compound (B1-2) is obtained, for example, by reacting the compound (B1-1) with magnesium to prepare a Grignard reagent, and reacting the Grignard reagent with zinc chloride or the like.

R¹³ and R¹⁴ in the compound (B2) are each independently an alkyl group, an alkoxy group, a hydroxyl group, or R¹³ and R¹⁴ are linked to form a ring structure. The alkyl group in R¹³ and R¹⁴ may be linear or may have a branched or ring structure. The number of carbon atoms in the alkyl group is preferably 1 to 30, more preferably 1 to 20, still more preferably 1 to 10, and particularly preferably 1 to 6 from the viewpoint of increasing the yield of the present production method. The alkoxy group in R¹³ and R¹⁴ can be represented by —OR²⁹, and R²⁹ is the same as the alkyl group in R¹³ and R¹⁴. In addition, the structure in which R¹³ and R¹⁴ are linked to form a ring structure can be represented by, for example, *—R²⁹—*. *—O—R²⁹—*, and *—O—R²⁹—O—*, and R²⁹ is as described above. Here, * is a linking group bonded to B in the formula (B2), and a ring structure containing B is formed.

When R¹³ or R¹⁴ is an alkyl group, R¹³ or R¹⁴ may be introduced instead of R¹¹ in the reaction in the present production method, and for example, the following compounds (C3) to (C9) and the like may be generated.

G¹-L¹-CR¹R²—R¹³ Formula (C7)

R¹¹—CR³R⁴-L²-G²-L³-CR⁵R⁶—R¹³ Formula (C8)

R¹³—CR³R⁴-L²-G²-L³-CR⁵R⁶—R¹³ Formula (C9)

where, each reference sign in the formula is as described above.

As in the case of using the compound (B1), these by-products may be separated according to the application of the compound (C1) or (C2) to be obtained, or may be used as a mixture. When the compound (B2) in which R¹³ and R¹⁴ are linked to form a ring structure is used, generation of by-products can be suppressed.

In the present production method, the amount of the compound (B1) and the compound (B2) used is preferably 0.5 equivalents to 30 equivalents, more preferably 1 equivalent to 20 equivalents, and still more preferably 1.5 equivalents to 10 equivalents, relative to the total amount of X¹ to X³ contained in the compound (A1) or the compound (A2), from the viewpoint of improving the yield of the target product.

The transition metal compound can be appropriately selected and used from known catalysts. As the transition metal compound, a compound containing elements from groups 3 to 12 of the periodic table as transition metals is preferable, and among them, a compound containing elements from groups 8 to 11 is preferable. Among them, the elements from groups 8 to 11 preferably include one or more elements selected from nickel and palladium.

When the transition metal compound contains nickel, the nickel may be any of a zerovalent compound, a monovalent compound, a divalent compound, and a trivalent compound, and among them, zerovalent or divalent nickel salts or complex salts are preferable from the viewpoint of catalytic ability and stability. Further, nickel chloride (NiCl₂) is more preferable from the viewpoint of easy availability and the like. In addition, nickel chloride may be an anhydride or a hydrate, but nickel chloride anhydride is more preferable from the viewpoint of catalytic ability.

When the transition metal compound contains palladium, the palladium may be any of a zerovalent compound and a divalent compound, and among them, zerovalent or divalent palladium salts or complex salts are preferable from the viewpoint of catalytic ability and stability. Furthermore, tris(dibenzylideneacetone)dipalladium (Pd₂(dba)₃) and palladium acetate (Pd(OAc)₂) are more preferable from the viewpoint of easy availability and the like. In addition, tris(dibenzylideneacetone)dipalladium and palladium acetate may be anhydrides or hydrates, but tris(dibenzylideneacetone)dipalladium anhydride and palladium acetate anhydride are more preferable from the viewpoint of catalytic ability.

The amount of the transition metal compound used is, for example, 0.05 to 50 equivalents, preferably 0.1 to 30 equivalents, more preferably 0.15 to 20 equivalents, relative to the total amount of 1 that the compound (A1) or the compound (A2) has.

In the reaction of the present production method, a ligand may be used in combination with a transition metal compound as a catalyst as necessary. By using the ligand, the yield of the target product is improved. On the other hand, in the present production method, since a sufficient yield can be obtained without using a ligand, the ligand may not be used.

Examples of the ligand include 1,3-butadiene, tricyclohexylphosphine, 1,1-bis(diphenylphosphino)ferrocene phenylpropyne, tetramethylethylenediamine (TMEDA), Sphos, Xphos, and Xantphos. When a ligand is used, the amount used is preferably 0.01 and 5.0 equivalents, more preferably 0.1 to 3.0 equivalents, relative to the metal catalyst used from the viewpoint of improving the yield of the target product.

In the reaction of the present production method, a base may be used as necessary. By using the ligand, the yield of the target product is improved. Examples of the base include potassium phosphate.

When a base is used, the amount used is preferably 0.1 and 20.0 equivalents, more preferably 1 to 20 equivalents, relative to the total number of 1 of the compound (A1) or the compound (A2) from the viewpoint of improving the yield of the target product.

In addition, the reaction of the present production method is usually performed in a solvent. The solvent can be appropriately selected and used from among solvents capable of dissolving the compound (A1) or the compound (A2) and the compound (B1) or the compound (B2). The solvent may be a single solvent or a mixed solvent of two or more solvents.

For example, the solvent is not particularly limited as long as the solvent is a solvent inert to the reaction, and as the solvent inert to the reaction, particularly, an ether-based solvent such as diethyl ether, tetrahydrofuran (THF), or dioxane is preferable, and tetrahydrofuran is more preferable.

In addition, for compounds having a relatively high fluorine atom content, such as the compound (A1) and the compound (A2), a fluorine-based solvent is more preferable, and a mixed solvent obtained by combining the ether-based solvent and the fluorine-based solvent is still more preferable.

Examples of the fluorine-based solvent include hydrofluorocarbons (1H,4H-perfluorobutane, 1H-perfluorohexane, 1,1,1,3,3-pentafluorobutane, 1,1,2,2,3,3,4-heptafluorocyclopentane, 2H,3H-perfluoropentane, and the like), hydrochlorofluorocarbons (3,3-dichloro-1,1,1,2,2-pentafluoropropane, 1,3-dichloro-1,1,2,2,3-pentafluoropropane (HCFC-225cb), and the like), hydrofluoroethers (CF₃CH₂OCF₂CF₂H(AE-3000), (perfluorobutoxy)methane, (perfluorobutoxy)ethane, and the like), hydrochlorofluoroolefins ((Z)-1-chloro-2,3,3,4,4,5,5-heptafluoro-1-pentene (HCFO-1437 dycc (Z) form), (E)-1-chloro-2,3,3,4,4,5,5-heptafluoro-1-pentene (HCFO-1437 dycc (E) form), (Z)-1-chloro-2,3,3-trifluoro-1-propene (HCFO-1233 yd (Z) form), (E)-1-chloro-2,3,3-trifluoro-1-propene (HCFO-1233 yd (E) form), and the like), and fluorine-containing aromatic compounds (perfluorobenzene, m-bis(trifluoromethyl)benzene(SR-solvent), p-bis(trifluoromethyl)benzene, and the like). Among them, hydrofluoroethers (CF₃CH₂OCF₂CF₂H(AE-3000)) are preferable as the fluorine-based solvent.

In the first production method, for example, a solution containing the compound (A1) or the compound (A2) is prepared, a transition metal compound and a ligand are added as necessary, and then a separately prepared compound (B1) is added to obtain the compound (C1) or the compound (C2).

The reaction temperature between the compound (A1) or the compound (A2) and the compound (B1) may be appropriately adjusted according to the combination of the compound (A1) or the compound (A2) and the compound (B1). For example, the temperature may be −20° C. to 66° C. (boiling point of tetrahydrofuran), and is preferably −25° C. to 60° C.

### <Second Production Method>

A second production method of a fluorine-containing compound of the present invention is a method for producing a fluorine-containing compound represented by the following formula (C1) or (C2), the method including: reacting a compound represented by the following formula (A3) or (A4) with the following formula (B3).

G¹-L¹-CR¹R²—ZnR¹² Formula (A3)

R¹²Zn—CR³R⁴-L²-G²-L³-CR⁵R⁶—ZnR¹² Formula (A4)

R¹¹—X⁴ Formula (B3)

G¹-L¹-CR¹R²—R¹¹ Formula (C1)

R¹¹—CR³R⁴-L²-G²-L³-CR⁵R⁶—R¹¹ Formula (C2)

where, X⁴ is a halogen atom, and preferred embodiments are the same as X¹ to X³. In addition, other reference signs in the formula are the same as those described in the first production method, and preferred embodiments are also the same.

The second production method is a method of synthesizing the compound (C1) or the compound (C2) by subjecting the organozinc compound (A3) or the compound (A4) having a fluoroalkyl chain or a (poly)oxyfluoroalkylene chain and the organohalogen compound (B3) having a substituent R¹¹ to a coupling reaction. In addition, the second production method is different from the first production method in that the compound (A3) and the compound (A4) having a fluoroalkyl chain or a (poly)oxyfluoroalkylene chain are organozinc compounds, and the compound (B3) having a substituent R¹¹ to be introduced is an organohalogen compound. Hereinafter, the second production method will be described, but the description of contents common to the first production method will be omitted here.

Preferred specific examples of the compound (A3) and the compound (A4) include the following compounds.

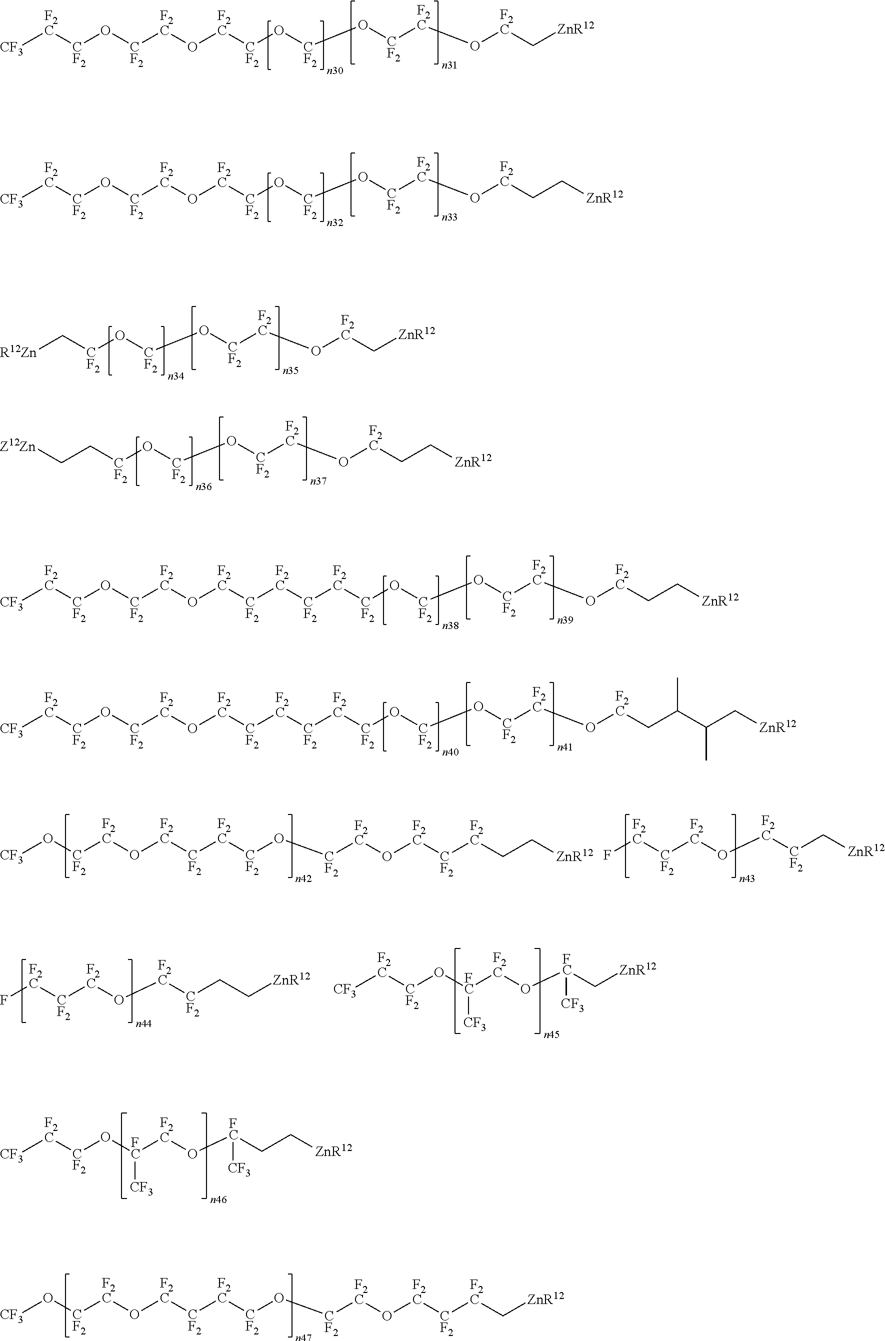

where, R¹² is as described above, and n30 to n47 represent the number of repeating units, and each independently represent an integer of 1 to 200.

The compound (B3) can be produced, for example, by a method of reacting the compound represented by the following formula (B3-1) with triphenylphosphine and iodomethane to iodinate the compound, a method of reacting the compound with triphenylphosphine and iodine to iodinate the compound, or the like. In addition, a commercially available product having a desired structure may be used.

R¹¹—OH Formula (B3-1)

where, R¹¹ in the formula is as described above.

Preferred specific examples of the compound (B3) include the following compounds.

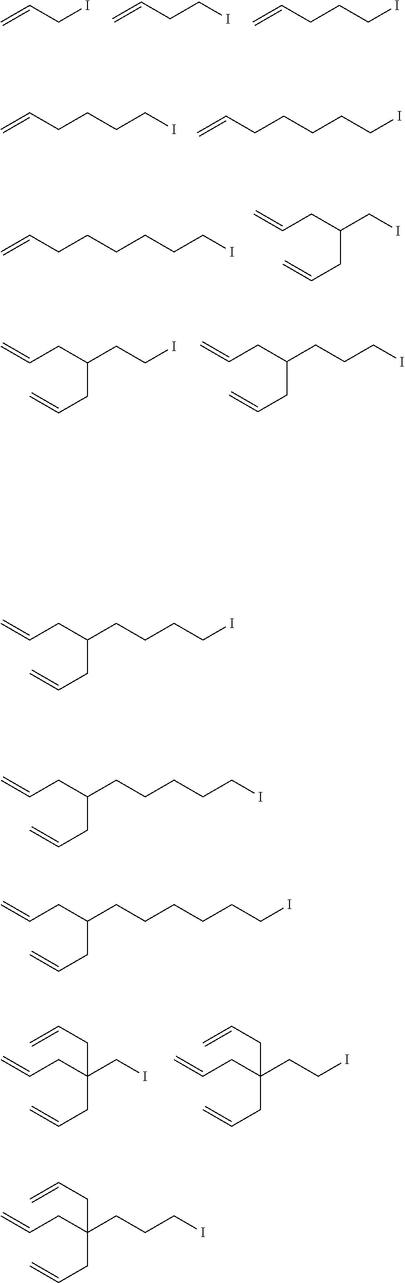

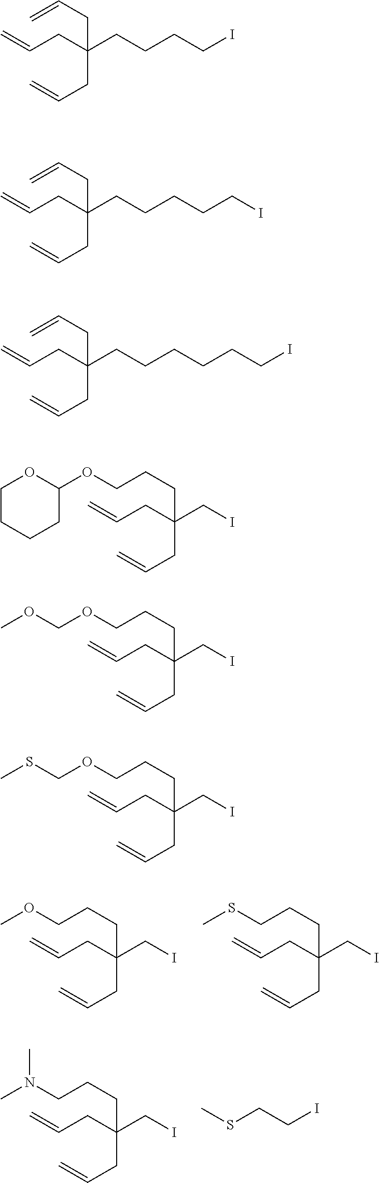

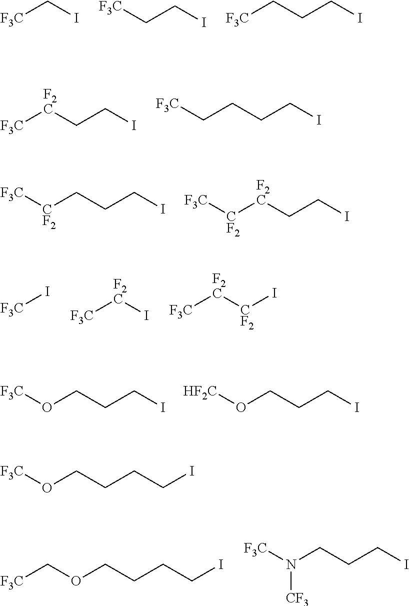

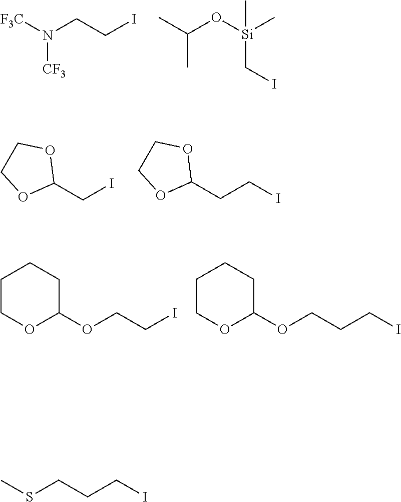

The ratio of the solvent, the catalyst, and the raw material, the reaction temperature, and the like in the second production method can be the same as those in the first production method, and preferred embodiments are also the same.

As described above, the compound (C1) or the compound (C2) in which various substituents are introduced into the (poly)oxyfluoroalkylene chain is obtained by the present production method. Fluorine-containing compounds are excellent in various properties such as low refractive index, low dielectric constant, water/oil repellency, heat resistance, chemical resistance, chemical stability, and transparency, and can be used in various fields such as electric and electronic materials, semiconductor materials, optical materials, and surface treatment agents.

In addition, a surface treatment agent can also be produced by introducing a reactive silyl group into the compound (C1) or the compound (C2) obtained by the present production method. The fluorine-containing compound having a (poly)oxyfluoroalkylene chain and a hydrolyzable silyl group can form a surface layer exhibiting high lubricity, water/oil repellency, and the like on the surface of the substrate, and thus is suitably used as a surface treatment agent.

The method for introducing a reactive silyl group into the compound (C1) or (C2) may be appropriately selected according to the substituent of the compound (C1) or the compound (C2). As an example, when the compound (C1) or the compound (C2) has a double bond, the compound (C1) or the compound (C2) can be introduced by subjecting the double bond and the following compound (E1) or (E2) to a hydrosilylation reaction.

HSi(R⁴⁰)_{3-c}(L)_{c} Formula (E1)

HSi(R⁴¹)₃₋ₖ[—(OSi(R⁴²)₂)ₚ—O—Si(R⁴⁰)_{3-c}(L)_{c}]ₖ Formula (E2)

where, in the formula,
R⁴⁰ is an alkyl group, and when there are a plurality of R⁴⁰'s, the R⁴⁰'s may be the same as or different from each other,
L is a hydrolyzable group or a hydroxyl group, and a plurality of L's may be the same as or different from each other,
R⁴¹ is an alkyl group, and when there are a plurality of R⁴¹'s, the R⁴¹'s may be the same as or different from each other,
R⁴² is an alkyl group, a phenyl group, or an alkoxy group, and two R⁴²'s may be the same or different;
c is 2 or 3,
k is 2 or 3, and
p is an integer of 0 to 5, and when p is two or more, two or more (OSi(R⁴²)₂)'s may be the same as or different from each other.

In addition, the compound (E2) can be produced, for example, by the method described in the specification of International Patent Publication No. WO 2019/208503.

In the compounds (C1) and (C2), when R¹¹ is a group represented by the formula (D1), a surface treatment agent represented by the following formula is obtained.

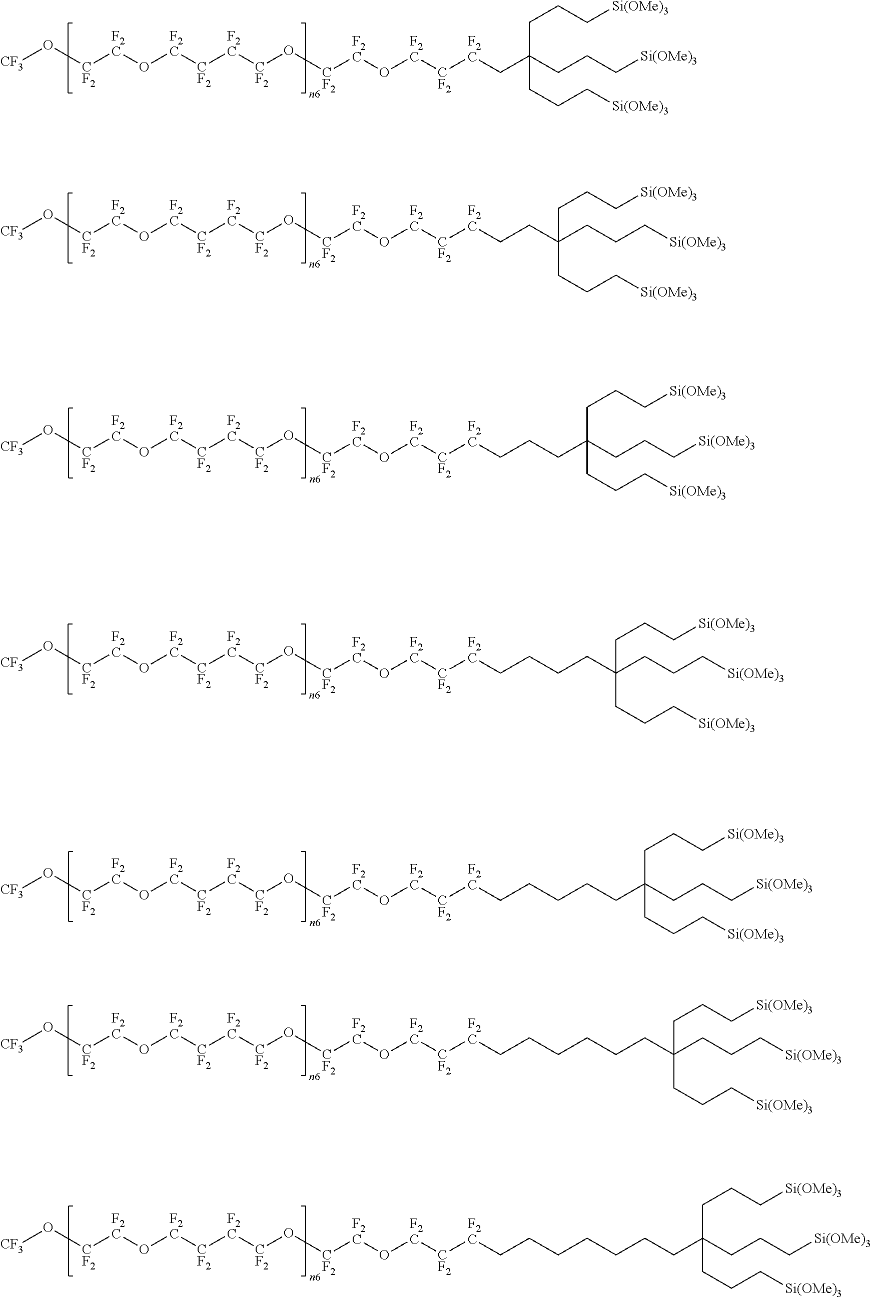

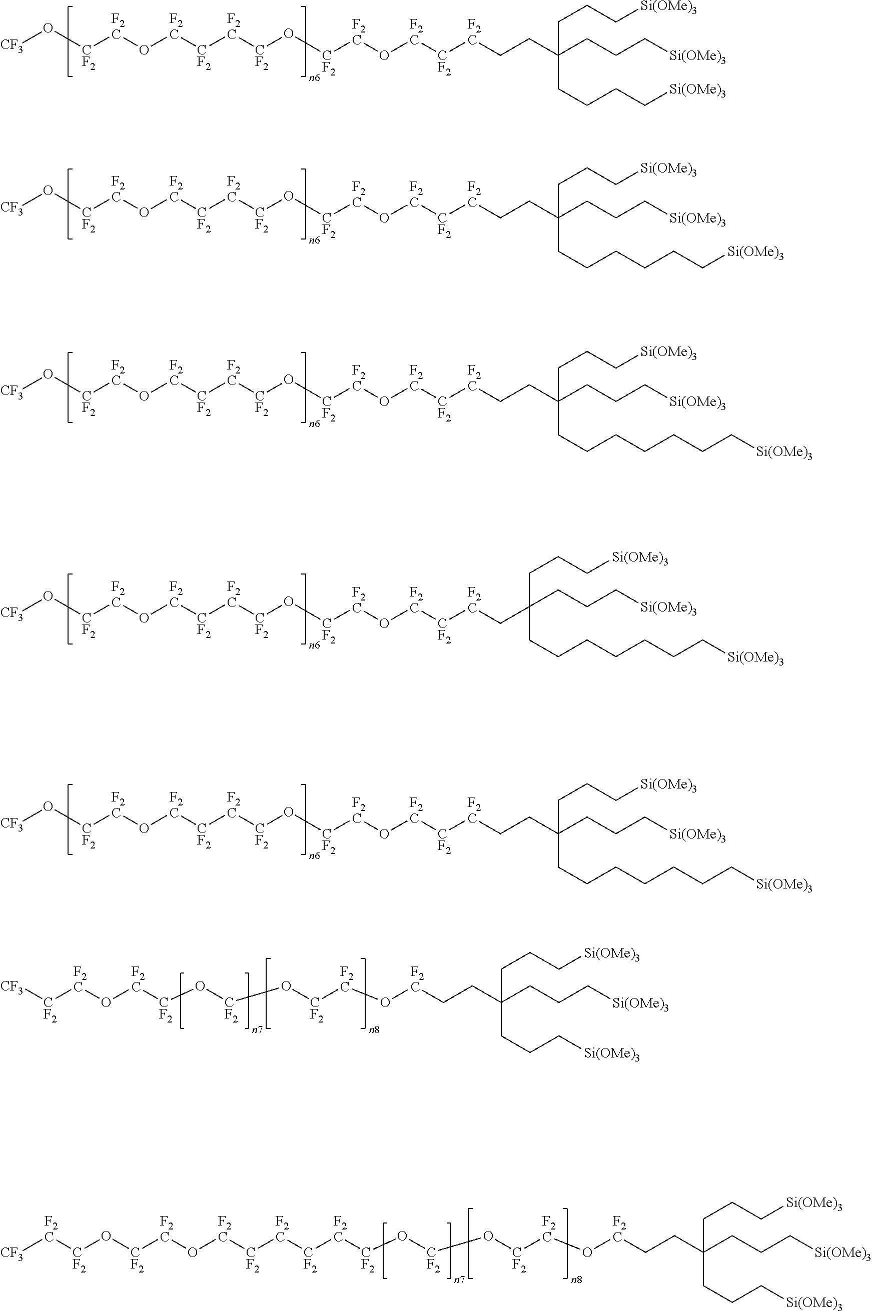

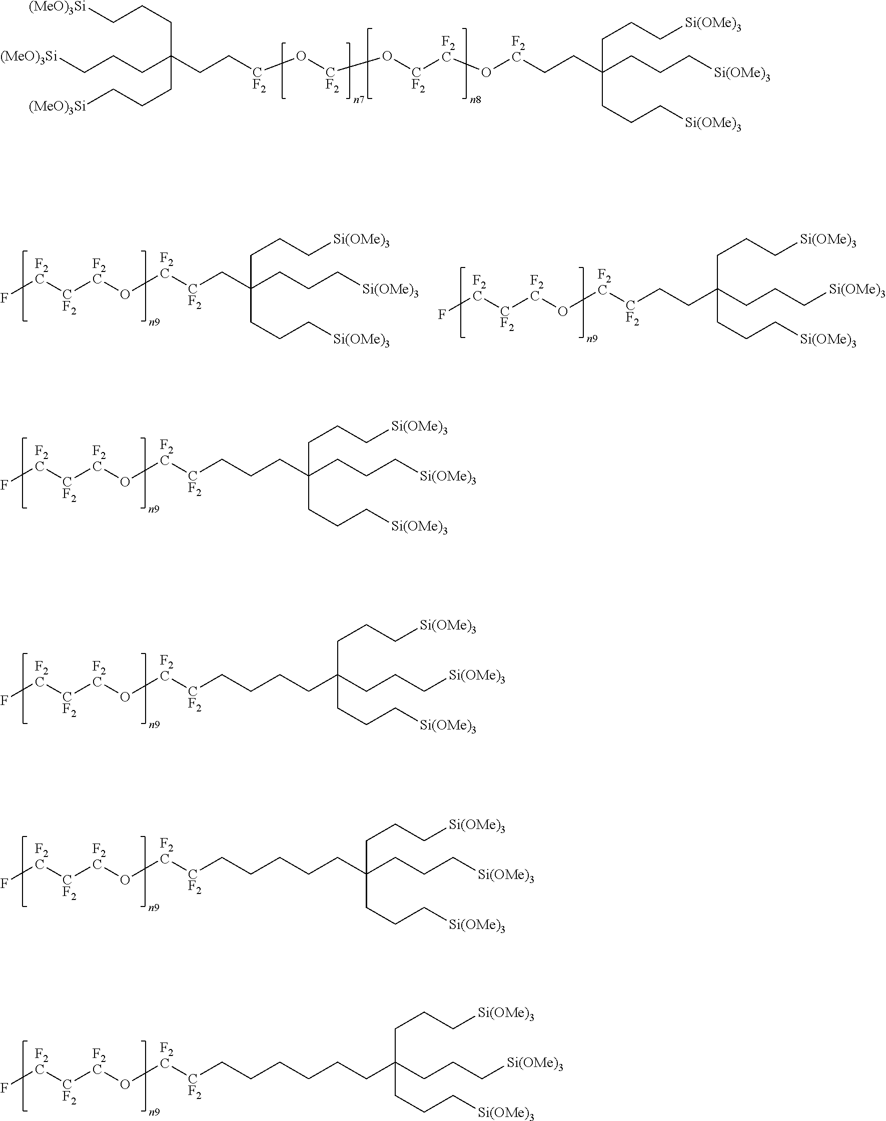

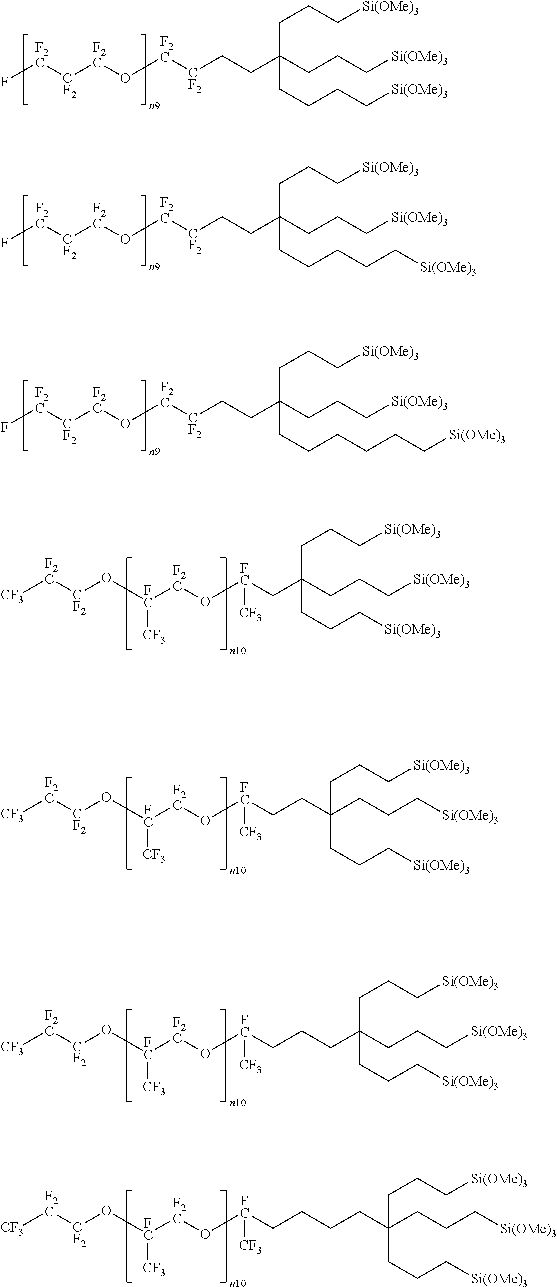

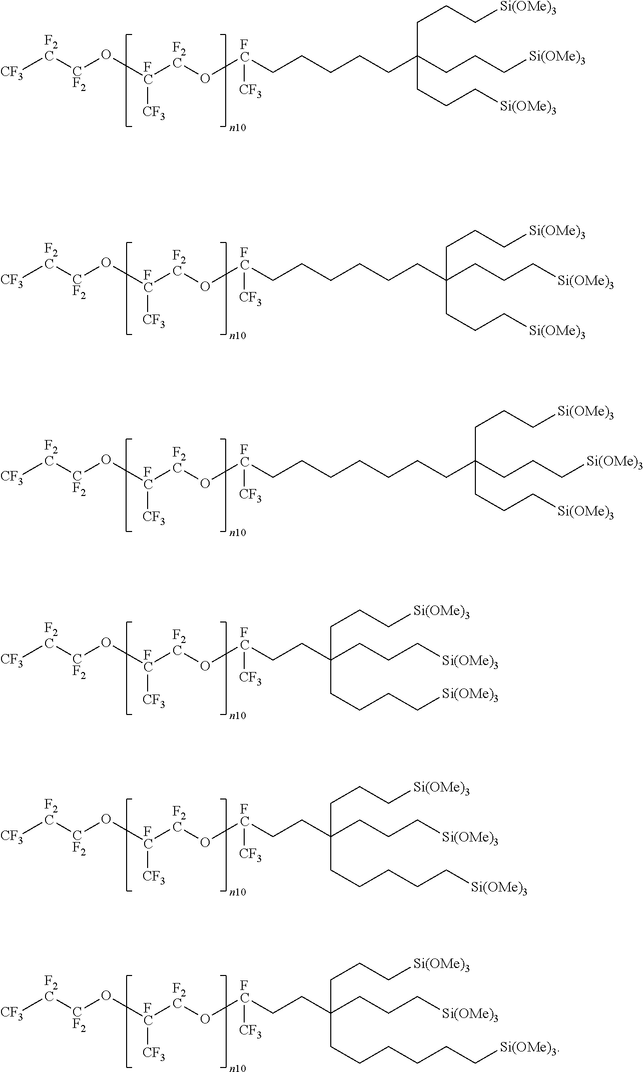

In addition, n6 to n10 in the formula represent the number of repeating units, and each independently represent an integer of 1 to 200.

The reactive silyl group is a group in which one or both of a hydrolyzable group and a hydroxyl group are bonded to a silicon atom. The hydrolyzable group is a group that becomes a hydroxyl group by a hydrolysis reaction. That is, the hydrolyzable silyl group becomes a silanol group (Si—OH) by a hydrolysis reaction. The silanol group further undergoes a dehydration condensation reaction between molecules to form a Si—O—Si bond. In addition, the silanol group undergoes a dehydration condensation reaction with a hydroxyl group (substrate —OH) on the surface of the substrate to form a chemical bond (substrate —O—Si).

Examples of the hydrolyzable group include an alkoxy group, a halogen atom, an acyl group, and an isocyanate group. The alkoxy group is preferably an alkoxy group having 1 to 6 carbon atoms. The halogen atom is preferably a chlorine atom.

The hydrolyzable group is preferably an alkoxy group or a halogen atom from the viewpoint of ease of production. As the hydrolyzable group, an alkoxy group having 1 to 4 carbon atoms is preferable from the viewpoint of having less outgassing at the time of coating and being excellent in storage stability of the present compound, an ethoxy group is particularly preferable when long-term storage stability of the present compound is required, and a methoxy group is particularly preferable when the reaction time after coating the substrate with the surface treatment agent is shortened.

Examples of the substrate include substrates required to be imparted with water/oil repellency. Examples thereof include other articles (for example, stylus), substrates that may be used by being brought into contact with human fingers, substrates that may be held by human fingers during operation, and substrates that may be placed on other articles (for example, placing tables).

Examples of the material of the substrate include metal, resin, glass, sapphire, ceramic, stone, and composite materials thereof. The glass may be chemically strengthened. A base film such as a SiO₂ film may be formed on the surface of the substrate.

As the substrate, a substrate for a touch panel, a substrate for a display, and a spectacle lens are preferred, and a substrate for a touch panel is particularly preferred. The material of the substrate for a touch panel is preferably glass or a transparent resin.

In addition, as the substrate, glass or a resin film used for an exterior part (excluding a display unit) in a device such as a mobile phone (for example, a smartphone), a portable information terminal (for example, a tablet terminal), a game machine, or a remote controller is also preferable.

The surface treatment agents containing the fluorine-containing compound are suitably used for applications in which it is required to maintain performance (friction resistance) in which water/oil repellency is less likely to decrease even when the surface layer is repeatedly rubbed with a finger and performance (fingerprint dirt removability) in which a fingerprint attached to the surface layer can be easily removed by wiping, for a long period of time, for example, as a surface treatment agent for a member constituting a surface touched by a finger of a touch panel, a spectacle lens, and a display of a wearable terminal.

### EXAMPLES

Hereinafter, the present invention will be described in detail using examples, but the present invention is not limited to these examples. Note that Examples 1 to 15 are examples.

### Example 1

### Synthesis Example 1-1: Synthesis of Compound (1-1)

The following compound (1-1) was obtained according to the method described in Example 11 of International Patent Publication No. WO 2013/121984.

CF₃—O—(CF₂CF₂O—CF₂CF₂CF₂CF₂O)n(CF₂CF₂O)—CF₂CF₂CF₂—CH₂CH₂I Formula (1-1)

The average value of the number of repeating units n is 13.

### Synthesis Example 1-2: Synthesis of Compound (2-1)

Diethyl Diallylmalonate (60.0 g), lithium chloride (23.7 g), water (6.45 g), and dimethyl sulfoxide (263 g) were added, and the mixture was stirred at 160° C. After cooling to room temperature, water was added, and the mixture was extracted with ethyl acetate. Hexane was added to the organic layer, washed with saturated saline, and dried over sodium sulfate. After the filtration, the solvent was distilled off to obtain 39.5 g of the following compound (2-1).

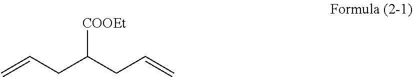

NMR spectrum of the compound (2-1);

¹H-NMR (400 MHz, Chloroform-d) δ (ppm): (ddt, J=17.1, 10.1, 7.0 Hz, 2H), 5.06 to 4.94 (m, 4H), 4.09 (q, J=7.1 Hz, 2H), 2.47 (ddd, J=14.0, 8.0, 6.1 Hz, 1H), 2.33 (dt, J=14.9, 7.5 Hz, 2H), 2.22 (dt, J=14.1, 6.5 Hz, 2H), 1.21 (t, J=7.1 Hz, 3H).

### Synthesis Example 1-3: Synthesis of Compound (2-2)

THF (260 mL) and diisopropylamine (29.8 mL) were added, and then the solution was cooled to −78° C. An n-butyl lithium hexane solution (2.76 M, 96.6 mL) was added, and the temperature was raised to 0° C. After stirring, the mixture was cooled to −78° C. to prepare a THF solution of lithium diisopropylamide (LDA). The compound (2-1) (39.5 g) was added to the THF solution, and the mixture was stirred, then allyl bromide (24.1 mL) was added thereto. The temperature was raised to 0° C., 1 M hydrochloric acid (100 mL) was added, and THF was distilled off under reduced pressure. After extraction with dichloromethane, sodium sulfate was added. After the filtration, the solvent was distilled off, and flash column chromatography was performed using silica gel to obtain 45.0 g of the compound (2-2).

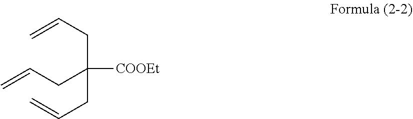

NMR spectrum of the compound (2-2);

¹H-NMR (400 MHz, Chloroform-d) δ (ppm): 5.74 to 5.62 (m, 3H), 5.04 (dd, J=13.6, 1.9 Hz, 6H), 4.10 (q, J=7.1 Hz, 2H), 2.29 (d, J=7.4 Hz, 6H), 1.22 (t, J=7.1 Hz, 3H).

### Synthesis Example 1-4: Synthesis of Compound (2-3)

The compound (2-2) (45.0 g) was dissolved in THF (620 mL), and the solution was cooled to 0° C. A THF solution (104 mL) of lithium aluminum hydride was added thereto, and the mixture was stirred. Water and a 15% sodium hydroxide aqueous solution were added thereto, the mixture was stirred at room temperature, and then diluted with dichloromethane. After the filtration, the solvent was distilled off, and flash column chromatography was performed using silica gel to obtain 31.3 g of the following compound (2-3).

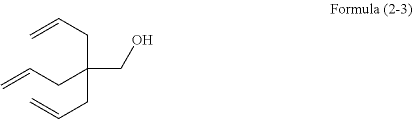

NMR spectrum of the compound (2-3);

¹H-NMR (400 MHz, Chloroform-d) δ (ppm): 5.90 to 5.76 (m, 3H), 5.10 to 5.02 (m, 6H), 3.38 (s, 2H), 2.03 (dt, J=7.5, 1.2 Hz, 6H), 1.45 (s, 1H).

### Synthesis Example 1-5: Synthesis of Compound (3-1)

Acetonitrile (380 mL), the compound (2-3) (31.3 g), triphenylphosphine (64.3 g), and carbon tetrachloride (33.9 g) were added, and the mixture was stirred at 90° C. After concentration, ethyl acetate/hexane was added and stirred. After filtration and concentration, 28.2 g of the following compound (3-1) was obtained by distillation.

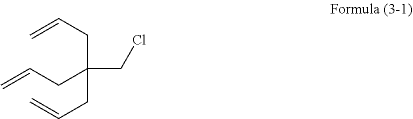

NMR spectrum of the compound (3-1);

¹H-NMR (400 MHz, Chloroform-d) δ (ppm): 5.83 to 5.67 (m, 3H), 5.16 to 5.01 (m, 6H), 3.32 (s, 2H), 2.05 (dt, J=7.5, 1.1 Hz, 6H).

### Synthesis Example 1-6: Synthesis of Organozinc Compound (3-2)

THF (35 mL) and iodine (0.180 g) were added to magnesium (2.36 g), and the mixture was stirred at room temperature. A THF (35 mL) solution of the compound (3-1) (14.0 g) was added thereto, and the mixture was heated and refluxed for 2 hours to prepare a Grignard reagent.

Zinc chloride (0.277 g) was added to the Grignard reagent (0.1 ml) derived from the above formula (3-1) prepared to be 17% by weight, and the mixture was stirred at room temperature for 2 hours. The reaction crude liquid was filtered through a glass filter, 1.4 dioxane (5.0 ml) was added to the filtrate, and the mixture was stirred at room temperature. The reaction crude liquid was filtered through a glass filter to obtain 8.2 g of solution containing the following compound (3-2).

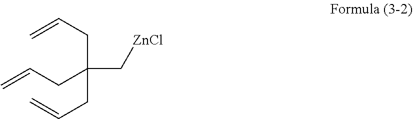

NMR spectrum of the compound (3-2);

¹H-NMR (400 MHz, Chloroform-d) δ (ppm): 5.88 (m, 3H), 5.11 (m, 6h), 1.85 (m, 6h), 1.22 (s, 2h).

### Synthesis Example 1-7: Synthesis of Compound (1-2)

CuCl₂ (7.0 mg), 1-phenyl-1-propyne (0.026 g), 1,3-bistrifluoromethylbenzene (23 mL) and the compound (1-1) (1.52 g) were added, and then the compound (3-2) (4.5 mL) was added. After stirring the mixture at room temperature, the mixture was washed with 1 M hydrochloric acid and dried over sodium sulfate. After filtration, the solvent was distilled off, and THF was added. After washing the mixture with DMF, flash column chromatography was performed using silica gel to obtain 0.210 g of the following compound (1-2).

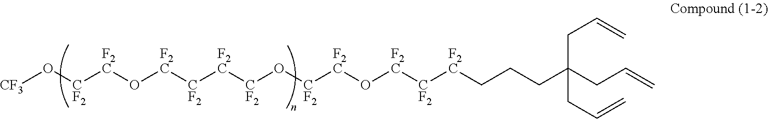

NMR spectrum of the compound (1-2);

¹H-NMR (400 MHz, Chloroform-d) δ (ppm): 5.80 (ddt, J=20.3, 9.3, 7.4 Hz, 3H), 5.01 (dd, J=13.5, 1.7 Hz, 6H), 2.13 to 2.01 (m, 2H), 1.97 (d, J=7.5 Hz, 6H), 1.67 to 1.55 (m, 2H), 1.27 to 1.18 (m, 2H).

¹⁹F-NMR (376 MHz, Chloroform-d) δ (ppm): −55.25, −82.83, −88.06, −90.16 (d, J=8.1 Hz), −114.18, −125.26, −126.59.

### Examples 2 to 9

The compound (1-2) was produced in the same manner as in Example 1, except that various conditions were changed as shown in the following Table 1 in Synthesis Example 1-7 of Example 1.

In addition, in Table 1, the equivalent is based on the compound (1-1). The raw material conversion rate is a rate at which the compound (1-1) is converted, and the target product selectivity is a rate at which the target compound (1-2) is selected from the compounds to be converted from the compound (1-1).

### Example 10

### Synthesis Example 10-1: Synthesis of Organoboron Compound (3-3)

THF (35 mL) and iodine (0.180 g) were added to magnesium (2.36 g), and the mixture was stirred at room temperature. A THF (35 mL) solution of the compound (3-1) (14.0 g) was added thereto, and the mixture was heated and refluxed for 2 hours to prepare a solution (0.80 M) of the following compound (3-3).

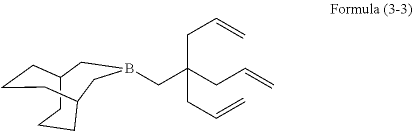 [SMILES: C=CCC(CC=C)(CC=C)CB1CC2CCCC(CCC2)C1]

NMR spectrum of the compound (3-3);

¹H-NMR (400 MHz, Chloroform-d) δ (ppm): 5.87 (m, 3H), 5.11 (m, 6H), 1.85-1.13 (m, 6H), 1.20-1.47 (m, 18H).

### Synthesis Example 10-2: Synthesis of Compound (1-2)

The compound (1-2) was produced in the same manner as in Synthesis Example 1-7 except that the compound (3-2) was changed to the compound (3-3) in Synthesis Example 1-7.

### Examples 11 to 15

The compound (1-2) was produced in the same manner as in Example 10-2, except that various conditions were changed as shown in the following Table 1 in Synthesis Example 10-2 of Example 10.

TABLE 1

| | Organometallic type | | | | | | |
|---|---|---|---|---|---|---|---|
| | Organoboron | | Catalyst (eq) | | Ligand | | Base |
| | compound or | | Compound | | Compound | | Compound |
| Example | organozinc | Equivalent | type | Equivalent | type | Equivalent | type |
| 1 | Compound (3-2) | 3 | NiCl₂ | 1 | 1.3-butadiene | 3 | None |
| 2 | Compound (3-2) | 3 | NiCl₂ | 1 | 1.3-butadiene | 3 | None |
| 3 | Compound (3-2) | 3 | Pd₂(dba)₃ | 0.2 | 1.3-butadiene | 1.5 | None |
| 4 | Compound (3-2) | 3 | Pd₂(dba)₃ | 0.2 | 1.3-butadiene | 2.1 | None |
| 5 | Compound (3-2) | 3 | Pd₂(dba)₃ | 0.1 | Sphos | 0.3 | None |
| 6 | Compound (3-2) | 3 | Pd₂(dba)₃ | 0.1 | Xphos | 0.3 | None |
| 7 | Compound (3-2) | 4 | Pd₂(dba)₃ | 0.1 | Xantphos | 0.3 | None |
| 8 | Compound (3-2) | 5.2 | Pd₂(dba)₃ | 0.1 | None | — | None |
| 9 | Compound (3-2) | 3 | None | — | — | — | None |
| 10 | Compound (3-3) | 2.8 | Pd(OAc)₂ | 0.3 | PCy₃ | 0.3 | K3PO4 |
| 11 | Compound (3-3) | 3.2 | Pd(OAc)₂ | 0.3 | PCy₃ | 0.7 | K3PO4 |
| 12 | Compound (3-3) | 3.4 | Pd(OAc)₂ | 0.9 | PCy₃ | 2.1 | K3PO4 |
| 13 | Compound (3-3) | 4 | Pd(OAc)₂ | 1 | PCy₃ | 2.8 | K3PO4 |
| 14 | Compound (3-3) | 2.1 | Pd(OAc)₂ | 0.5 | PCy₃ | 0.8 | K3PO4 |
| 15 | Compound (3-3) | 1.7 | Pd(OAc)₂ | 0.6 | PCy₃ | 0.8 | K3PO4 |

| | | | | | | Target |
|---|---|---|---|---|---|---|
| | | | | Reaction | Raw material | product |
| | Base | | Temperature | time | conversion rate | selectivity |
| Example | Equivalent | Catalyst | [° C.] | [h] | [%] | [%] |
| 1 | — | AB-3000 | 55 | 4 | 100 | 80 |
| 2 | — | THF | 55 | 4 | 100 | 81 |
| 3 | — | AB-3000 | 55 | 4 | 87 | 41 |
| 4 | — | THF | 55 | 4 | 63 | 59 |
| 5 | — | AB-3000 | 55 | 24 | 100 | 25 |
| 6 | — | AE-3000 | 55 | 24 | 100 | 26 |
| 7 | — | AB-3000 | 60 | 24 | 100 | 38 |
| 8 | — | AE-3000 | 60 | 24 | 100 | 20 |
| 9 | — | AE-3000 | 55 | 4 | 80 | 43 |
| 10 | 2.9 | THF | rt | 20 | 23 | 47 |
| 11 | 3.4 | AB-3000 | rt | 20 | 26 | 40 |
| 12 | 5.9 | THF | 60 | 20 | 45 | 39 |
| 13 | 10 | AB-3000 | 60 | 20 | 49 | 52 |
| 14 | 3.4 | THF | 80 | 17 | 100 | 14 |
| 15 | 4.1 | AB-3000 | 80 | 17 | 31 | 52 |

In addition, abbreviations and the like in Table 1 are as follows.
Pd₂(dba)₃: tris(dibenzylideneacetone)dipalladium
Pd(OAc)₂: palladium acetate
Sphos: 2-dicyclohexylphosphino-2′,6′-dimethoxybiphenyl
Xphos: 2-dicyclohexylphosphino-2′,4′,6′-triisopropylbiphenyl
Xantphos: 4,5-bis(diphenylphosphino)-9,9-dimethylxanthene
PCy₃: tricyclohexylphosphine
AE-3000: CF₃CH₂OCF₂CF₂H
THF: tetrahydrofuran
rt: room temperature (25° C.)

According to the present production method, any substituent can be introduced into a compound having a fluoroalkylene chain or a (poly)oxyfluoroalkylene chain under relatively mild reaction conditions using an easily available compound. The compound obtained by the present production method can be suitably used as, for example, a surface treatment agent capable of forming a surface layer having water/oil repellency, fingerprint wipe-off removability, and the like on a surface of a substrate, or a raw material thereof.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A method for producing a fluorine-containing compound represented by the following formula (C1) or (C2), the method comprising: reacting a compound represented by the following formula (A1) or (A2) with a compound represented by the following formula (B1) or (B2):
G¹-L¹-CR¹R²—X¹ Formula (A1)
X²—CR³R⁴-L²-G²-L³-CR⁵R⁶—X² Formula (A2)
R¹¹—ZnR¹² Formula (B1)
R¹¹—BR¹³R¹⁴ Formula (B2)
G¹-L¹-CR¹R²—R¹¹ Formula (C1)
R¹¹—CR³R⁴-L²-G²-L³-CR⁵R⁶—R¹¹ Formula (C2)
where, in the formula,
G¹ is a fluoroalkyl group or a monovalent group having a (poly)oxyfluoroalkylene chain,
G² is a fluoroalkylene group or a divalent group having a (poly)oxyfluoroalkylene chain,
L¹, L², and L³ are each independently a single bond or a divalent organic group,
R¹, R², R³, R⁴, R⁵, and R⁶ are each independently a hydrogen atom or an alkyl group having 1 to 6 carbon atoms which may have a substituent,
R¹¹ is a hydrocarbon group which may have a substituent or a heteroatom, and when there are a plurality of R¹¹'s, the R¹¹'s may be the same as or different from each other,
R¹² is a halogen atom or a hydrocarbon group which may have a substituent or a heteroatom,
R¹³ and R¹⁴ are each independently an alkyl group, an alkoxy group, and a hydroxyl group, or R¹³ and R¹⁴ are linked to form a ring structure, and
X¹, X², and X³ are each independently a halogen atom.

2. A method for producing a fluorine-containing compound represented by the following formula (C1) or (C2), the method comprising: reacting a compound represented by the following formula (A3) or (A4) with the following formula (B3):
G¹-L¹-CR¹R²—ZnR¹² Formula (A3)
R¹²Zn—CR³R⁴-L²-G²-L³-CR⁵R⁶—ZnR¹² Formula (A4)
R¹¹—X⁴ Formula (B3)
G¹-L¹-CR¹R²—R¹¹ Formula (C1)
R¹¹—CR³R⁴-L²-G²-L³-CR⁵R⁶—R¹¹ Formula (C2)
where, in the formula,
G¹ is a fluoroalkyl group or a monovalent group having a (poly)oxyfluoroalkylene chain,
G² is a fluoroalkylene group or a divalent group having a (poly)oxyfluoroalkylene chain,
L¹, L², and L³ are each independently a single bond or a divalent organic group,
R¹, R², R³, R⁴, R⁵, and R⁶ are each independently a hydrogen atom or an alkyl group having 1 to 6 carbon atoms which may have a substituent,
R¹¹ is a hydrocarbon group which may have a substituent or a heteroatom, and when there are a plurality of R¹¹'s, the R¹¹'s may be the same as or different from each other,
R¹² is a halogen atom or a hydrocarbon group which may have a substituent or a heteroatom, and when there are a plurality of R¹²'s, the R¹²'s may be the same as or different from each other, and
X⁴ is a halogen atom.

3. The method for producing a fluorine-containing compound according to claim 1, wherein at least one of the L¹-CR¹R², the L²-CR³R⁴, and the L³-CR⁵R⁶ is represented by (CR⁷R⁸—R⁹R¹⁰)ₙ₁,
where, in the formula,
R⁷, R⁸, R⁹, and R¹⁰ are each independently a hydrogen atom or an alkyl group having 1 to 6 carbon atoms, which may have a substituent, and when there are a plurality of R⁷'s, R⁸'s, R⁹'s, and R¹⁰'s, the R⁷'s, R⁸'s, R⁹'s, and R¹⁰'s may be the same as or different from each other, and
n1 is an integer of 1 to 20.

4. The method for producing a fluorine-containing compound according to claim 2, wherein at least one of the L¹-CR¹R², the L²-CR³R⁴, and the L³-CR⁵R⁶ is represented by (CR⁷R⁸—R⁹R¹⁰)ₙ₁,
where, in the formula,
R⁷, R⁸, R⁹, and R¹⁰ are each independently a hydrogen atom or an alkyl group having 1 to 6 carbon atoms, which may have a substituent, and when there are a plurality of R⁷'s, R⁸'s, R⁹'s, and R¹⁰'s, the R⁷'s, R⁸'s, R⁹'s, and R¹⁰'s may be the same as or different from each other, and
n1 is an integer of 1 to 20.

5. The method for producing a fluorine-containing compound according to claim 1, wherein at least one of the L¹-CR¹R², the L²-CR³R⁴, and the L³-CR⁵R⁶ is represented by (CH₂CH₂)ₙ₂,
where, in the formula, n2 is an integer of 1 to 20.

6. The method for producing a fluorine-containing compound according to claim 2, wherein at least one of the L¹-CR¹R², the L²-CR³R⁴, and the L³-CR⁵R⁶ is represented by (CH₂CH₂)ₙ₂,
where, in the formula, n2 is an integer of 1 to 20.

7. The method for producing a fluorine-containing compound according to claim 1, wherein R¹¹ is represented by the following formula (D1):
(CH₂═CH—R²¹—)ₐ(R²²—)₃₋ₐC—R²³—* Formula (D1)
where
R²¹ is a single bond or an alkylene group which may have a fluorine atom having 1 to 18 carbon atoms, and when there are a plurality of R²¹'s, the R²¹'s may be the same as or different from each other,
R²² is a hydrogen atom or an alkyl group which may have a fluorine atom having 1 to 10 carbon atoms, and when there are a plurality of R²²'s, the R²²'s may be the same as or different from each other,
R²³ is a single bond or an alkylene group having 1 to 19 carbon atoms, a is an integer of 1 to 3, and
* is an atomic bond.

8. The method for producing a fluorine-containing compound according to claim 2, wherein R¹¹ is represented by the following formula (D1):
(CH₂═CH—R²¹—)ₐ(R²²—)₃₋ₐC—R²³—* Formula (D1)
where
R²¹ is a single bond or an alkylene group which may have a fluorine atom having 1 to 18 carbon atoms, and when there are a plurality of R²¹'s, the R²¹'s may be the same as or different from each other,
R²² is a hydrogen atom or an alkyl group which may have a fluorine atom having 1 to 10 carbon atoms, and when there are a plurality of R²²'s, the R²²'s may be the same as or different from each other,
R²³ is a single bond or an alkylene group having 1 to 19 carbon atoms,
a is an integer of 1 to 3, and
* is an atomic bond.

9. The method for producing a fluorine-containing compound according to claim 1, wherein at least one of X¹, X², X³, and X⁴ is an iodine atom.

10. The method for producing a fluorine-containing compound according to claim 2, wherein at least one of X¹, X², X³, and X⁴ is an iodine atom.

11. The method for producing a fluorine-containing compound according to claim 1, wherein the reaction is performed in the presence of a transition metal compound.

12. The method for producing a fluorine-containing compound according to claim 2, wherein the reaction is performed in the presence of a transition metal compound.

13. The method for producing a fluorine-containing compound according to claim 11, wherein the transition metal compound contains one or more elements selected from Ni and Pd.

14. The method for producing a fluorine-containing compound according to claim 12, wherein the transition metal compound contains one or more elements selected from Ni and Pd.

15. A method for producing a surface treatment agent, comprising: producing a fluorine-containing compound represented by the formula (C1) or (C2) by the production method according to claim 1; and introducing a reactive silyl group into the fluorine-containing compound.

16. A method for producing a surface treatment agent, comprising: producing a fluorine-containing compound represented by the formula (C1) or (C2) by the production method according to claim 2; and introducing a reactive silyl group into the fluorine-containing compound.
